(19) United States
(12) Patent Application Publication
(10) Pub. No.: US 2008/0107603 A1
(43) Pub. Date: May 8, 2008

(54) Neuroprotectant methods, compositions, and screening methods thereof

(76) Inventor: Okezie I. Aruoma, London (GB)

Correspondence Address: DAVID A . JACKSON;KLAUBER & JACKSON, 4TH FLOOR, 411 HACKENSACK AVE., HACKENSACK, NJ, 07601, US

(21) Appl. No.: 11/347,016

(22) Filed: Feb. 3, 2006

Related U.S. Application Data
(63) Continuation of application No. PCT/US03/09840, filed on Mar. 28, 2003.
(60) Provisional application No. 60/367,845, filed on Mar. 28, 2002.

(51) Int. Cl. A61K 31/4164; A61K 31/122; A61K 31/455; A61K 31/70; A61K 31/7088; A61K 38/02; A61K 39/395; A61P 25/00

(52) U.S. Cl. 424/9.2; 424/130.1; 424/94.1; 514/2; 514/356; 514/398; 514/43; 514/44

(57) ABSTRACT

The present invention relates in general to methods of protecting a mammalian central nervous system cell from damage, and to methods of treating or ameliorating neurodegenerative diseases. The invention further relates to screening for neuro-protective agents that may alone, or in combination with other neuroprotective agents, aid in protecting cells of the central nervous system from damage attributed to neurotoxic compounds, free radicals, or neurodegenerative diseases. The invention further relates to pharmaceutical compositions comprising L-ergothioneine or other newly identified compounds and pharmaceutically acceptable carriers for administration to a mammal in need of neuroprotection.

### CROSS-REFERENCE TO RELATED APPLICATIONS

The present application claims priority to provisional application U.S. Ser. No. 60/367,845 filed Mar. 28, 2002, the disclosure of which is hereby incorporated by reference in its entirety. Applicants claim the benefit of the present application under 35 U.S.C. §119(e).

### FIELD OF THE INVENTION

The present invention relates in general to neuroprotective methods, and mor e specifically to methods for prevention of damage to cells of the central nervous system and methods for treatment of neurodegenerative diseases. Further, the invention provides for methods of screening for compounds capable of acting as neuroprotectants, and for pharmaceutical compositions useful for treating neurodegenerative diseases.

### BACKGROUND OF THE INVENTION

Neuronal degeneration as a result of Alzheimer's disease, multiple sclerosis, stroke, traumatic brain injury, spinal cord injuries, and other central nervous system disorders is an enormous medical and public health problem by virtue of both its high incidence and the frequency of long-term sequelae. Animal studies and clinical trials have shown that amino acid transmitters (especially glutamate), oxidative stress and inflammatory reactions contribute strongly to cell death in these conditions.

Upon injury or upon ischemic insult, damaged neurons release massive amounts of the neurotransmitter glutamate, which is excitotoxic to the surrounding neurons (Choi et al., (1988), Neuron 1:623-634; Rothman et al., (1984), J. Neurosci. 4:1884-1891; Choi and Rothman, (1990), Ann. Rev. Neurosci. 13:171-182; David et al., (1988), Exp. Eye Res. 46:657-662; Drejer et al., (1985), J. Neurosci. 45:145-151. See also U.S. Pat. No. 5,135,956 and U.S. Pat. No. 5,395,822, incorporated herein by reference in their entireties. Several studies have shown the involvement of glutamate in the pathophysiology of Huntington's disease (HD) (Coyle and Schwarcz, (1976), Nature 263:244-246, Alzheimer's disease (AD) (Maragos et al, (1987), TINS 10:65-68, epilepsy (Nadler et al, (1978), Nature 271:676-677, lathyrism (Spencer et al, (1986), Lancet 239:1066-1067, amyotropic lateral sclerosis (ALS) and Parkinsonian dementia of Guam (Calne et al, (1986), Lancet 2:1067-1070) as well as in the neuropathology associated with stroke, ischemia and reperfusion (Dykens et al, (1987), J. Neurochem. 49:1222-1228).

Thus, injury to neurons may be caused by overstimulation of receptors by excitatory amino acids including glutamate and aspartate (Lipton et al. (1994) New Engl. J. Med. 330:613-621). Indeed, the N-methyl-D-aspartate (NMDA) subtype of glutamate receptor is suggested to have many important roles in normal brain function, including synaptic transmission, learning and memory, and neuronal development (Lipston et al. (1994) supra; Meldrum et al. (1990) Trends Pharm. Sci. 11:379-387). However, over-stimulation of the NMDA subtype of glutamate receptor leads to increased free radical production and neuronal cell death, which can be modulated by antioxidants (Herin et al. (2001) J. Neurochem. 78:1307-1314; Rossato et al. (2002) Neurosci. Lett. 318:137-140).

Additionally, inflammation and oxidative stress are key components of the pathology of many chronic neurodegenerative conditions, including Alzheimer's disease (AD). Alzheimer's disease (AD) is characterized by the accumulation of neurofibrillary tangles and senile plaques, and a widespread progressive degeneration of neurons in the brain. Senile plaques are rich in amyloid precursor protein (APP) that is encoded by the APP gene located on chromosome 21. A commonly accepted hypothesis underlying pathogenesis of AD is that abnormal proteolytic cleavage of APP leads to an excess extracellular accumulation of beta-amyloid (Aβ) peptide that has been shown to be toxic to neurons (Selkoe et al., (1996), J. Biol. Chem. 271:487-498; Quinn et al., (2001), Exp. Neurol. 168:203-212; Mattson et al., (1997), Alzheimer's Dis. Rev. 12:1-14; Fakuyama et al., (1994), Brain Res. 667:269-272).

Parkinson's disease (PD) is a progressive neurodegenerative disorder characterized by a dysfunction of movement consisting of akinesia, rigidity, tremor and postural abnormalities. This disease has been associated with the loss of nigro-striatal dopaminergic neuronal integrity and functionality as evidenced by substantial loss of dopaminergic neurons in substantia nigra pars compacta (SNpc) (Pakkenberg et al. (1991) J. Neurol. Neurosurg. Psychiat. 54:30-33), and a decrease in content, synaptic and vesicular transporters of dopamine in the striatum (see, for example, Guttman et al. (1997) Neurology 48:1578-1583). The precise mechanisms for the loss of dopaminergic neurons may include a role for α-synuclein (Golbe (1999) Movement Discord 14:6-9), MAO-B (Mellick et al. (1999) Movement Discord 14:219-224) and CYP2D6 (Sabbagh et al. (1999) Movement Discord 14:230-236) mutations in a sub-population of familial PD, environmental factors in sporadic cases of PD (Gorell et al. (1998) Neurology 50:1346-1350), and oxidative stress in the more common idiopathic PD cases (see, for example, Olanow et al. (1999) Ann. Rev. Neurosci. 22:123-144). Hallmarks of the involvement of oxidative stress include iron deposition (see, for example, Sofic et al. (1991) J. Neurochem. 56:978-982), lipid peroxidation (Dexter et al. (1989) J. Neurochem. 52:381-389), protein oxidation (Alam et al. (1997) J. Neurochem. 69:1326-1329), DNA damage (see, for example, Alam et al., (1997) J. Neurochem. 69:1196-1203), decreased glutathione (GSH) levels (see, for example, Sian et al. (1994) Ann. Neurol. 36:356-361), increased superoxide dismutase levels (see, for example, Yoritaka et al. (1997) J. Neurol. Sci. 148:181-186) and associated low levels of antioxidants such as vitamin C and E, (de Rijk et al. (1997) Arch. Neurol. 54:762-765) arguing strongly for antioxidant prophylaxis in neurodegenerative disorders.

L-Ergothioneine (2-mercaptohistidine trimethylbetaine) (“ergothioneine”) (FIG. 1) is a sulphur-containing amino acid formed via hercynine from histidine, methionine and cysteine in microorganisms. L-Ergothioneine is not biosynthesized in animals, and thus is obtained only from dietary sources. Blood concentrations of ergothioneine in almost every species investigated are in near millimolar range (Table 1). The L-ergothioneine concentration in man is estimated to be in the range 46 μM to 183 μM.

TABLE 1
Blood concentration of L-Ergothioneine in various animals.

| | L-Ergothioneine Concentration |
|---|---|
| Species | (mg/100 ml blood) |
| Man | 1-4 |
| Rat | 1-6 |
| Rabbit | 1-10 |
| Guinea Pig | 1-4 |
| Cat | 0.5-2 |
| Dog | 3-6 |
| Ox | 0.5-2 |
| Pig | 3-2.7 |
| Sheep | 2-6 |
| Fowl | 2-10 |

### SUMMARY OF THE INVENTION

L-Ergothioneine (EGT) is radioprotective, antimutagenic, and scavenges singlet oxygen, hypochlorous acid, (HOCl), hydroxyl radicals, and peroxyl radicals (Hartman (1990) Meth. Enzymol. 259:310-318; Akanmu et al. (1991) Arch. Biochem. Biophys. 288:10-16). L-Ergothioneine inhibits peroxynitrite dependent nitration of the amino acid tyrosine and DNA, and confers cellular homeostasis in neuronal cells challenged with the mixture of N-acetyl cysteine/hydrogen peroxide (Aruoma et al. (1999) Fd. Chem. Toxicol. 37:1043-1053). L-ergothioneine also inhibits the formation of xanthine and hypoxanthine, which may have many implications for inflammatory conditions such as gout, a condition characterized by overproduction of uric acid (the oxidation product of xanthine) (Aruoma et al. (1999), Food Chem. Toxicology 37:1043-1053). However, molecular mechanisms underlying the chemoprotective effects of EGT remain largely unresolved.

One aspect of the present invention is directed to the neuroprotective effects of L-ergothioneine upon exogenous administration to neuronal cells to prevent the damaging effects of the glutamate agonist N-methyl-D-aspartate. Moreover, the present invention rests in part on the results of studies presented below which establish that the injection of glutamate agonist N-methyl-D-aspartate (NMDA) into the vitreous body of the rat eye results in a number of morphological changes in the retina. Most apparent was a dramatic reduction in the density and sizes of neurons accompanied by a decrease in amyloid precursor protein (APP) and glial fibrillary acid protein (GFAP) immunoreactivity. However, in animals treated with L-ergothioneine, cell loss was significantly reduced. Thus, the results establish that L-ergothioneine possesses the ability to protect neural cells from damage.

Further evidence of the neuroprotective effects of L-ergothioneine are shown in the present invention, wherein the neuroprotective effects of L-ergothioneine are documented in the 6-hydroxydopamine (6-OHDA) lesion rat model of Parkinson's disease (PD). As shown in the Example below, the number of tyrosine hydroxylase positive cells (TH+ cells) in the substantia nigra and striatal dopamine content in the vehicle treated rats were significantly decreased. Treatment of rats with L-ergothioneine before 6-OHDA lesioning markedly reduced the loss of both TH+ cells and striatal dopamine content. These data support the ability of L-ergothioneine to cross the blood-brain barrier and provide significant protection of striato-nigral integrity and functionality.

Accordingly, in a first aspect, the invention features a method of protecting a mammalian central nervous system (CNS) cell from damage, comprising administering a therapeutically effective amount of L-ergothioneine to a mammal in need thereof. In a more specific embodiment, the mammalian CNS cell is a neuronal cell and includes ganglion and non-ganglion cells including all of the biochemically defined neuronal populations such as the cholinergic, dopaminergic-and GABA (γ-aminobutyric acid)ergic neurons. In a more specific embodiment, the dopaminergic cells are tyrosine hydroxylase positive (TH+) cells of the substantia nigra. In one embodiment, the subject is a mammal; in a specific embodiment, the mammal is a human subject.

In a further specific embodiment, L-ergothioneine protects against neural damage resulting from (i) exposure to a neurotoxic compound, such as glutamate or a glutamate analog; other neurotoxic compounds may include certain anticancer compounds. (ii) exposure to one or more free radicals and oxidants such as, for example, singlet oxygen, hydroxyl radicals, peroxyl radicals, peroxynitrite, hydrogen peroxide, nitric oxide, hypochlorous acid (and other hypohalous acids) and/or metalloenzymes.

In yet a further embodiment, L-ergothioneine may protect against neural damage caused by the use of radiotherapy for treatment of certain cancers, including certain brain tumors, wherein the radiotherapy results in damage to cells and the release of free radicals and oxidants.

In another embodiment, L-ergothioneine may protect against neural damage caused by the presence of a neurodegenerative disease, such as for example, Alzheimer's disease, multiple sclerosis, Down's syndorome, amyotropic lateral sclerosis, Parkinson's disease, traumatic injury to neural tissue such as to the brain or spinal cord, macular degeneration, HIV/AIDS and optic neuropathies and retinopathies.

The method of the invention is useful with any mammal of interest. In a preferred embodiment, the mammal is a human being. A further embodiment would be for veterinary use in the treatment of domestic or non-domestic animals having suffered a traumatic injury.

In further embodiments, L-ergothioneine is administered as a dietary supplement in an amount effective to provide protection from neurotoxic compounds. In more specific embodiments, the dietary supplement is in the form of an oral capsule or tablet. In a yet further embodiment, L-ergothioneine may be administered sublingually or buccally.

In a further embodiment, L-ergothioneine is administered directly to the site of injury in an amount effective to inhibit the damage attributed to the release of free radicals and oxidants from injured cells and damaged tissue. In the case of a traumatic injury, such as a brain or spinal cord injury, L-ergothioneine may be delivered intrathecally, intraventricularly or intracranially.

In a related second aspect, the invention features a method of protecting a mammalian neural cell from neurodegeneration, comprising administering a therapeutically effective amount of L-ergothioneine to a mammal in need thereof. One specific embodiment includes a method of protecting a mammalian neural cell from neurodegeneration by administration of a pharmaceutical composition comprising L-ergothioneine and a pharmaceutically acceptable carrier. Such pharmaceutical compositions may be designed for oral delivery, intravenous delivery, intramuscular delivery, subcutaneous delivery, intrathecal delivery or intraventricular delivery. Certain embodiments may include specific carrier molecules that aid in L-ergothioneine crossing the blood brain barrier.

In the experiments described below, a retinal assay was used as an in vivo animal model to determine the neuroprotective capacity of L-ergothioneine. The retinal-vitreal model is useful for assessments of neurotoxicity and for identifying compounds able to protect neuronal cells from damage. The compounds identified by the screening method of the invention are useful to protect cells from neurodegenerative conditions and agents, for example, including their use for treatment and amelioration of neurodegeneration accompanying disease conditions such as Alzheimer's disease, multiple sclerosis, Down's syndrome, amyotropic lateral sclerosis, Parkinson's disease, traumatic injury including brain and spinal cord injury, macular degeneration, HIV/AIDS and optic neuropathies and retinopathies. Corroboration of the neuroprotective effects of L-ergothioneine were also demonstrated in the 6-OHDA animal model of Parkinson's disease, described below.

Accordingly, in a third aspect, the invention features a screening method for identifying compounds capable of protecting central nervous system cells from damage, comprising (a) exposing (treating) retinal neurons to neurotoxic agents with and without treatment with test compounds; and (b) determining the effect of the test compounds on-retinal neuron populations, wherein test compounds capable of increasing neuronal integrity are identified as neuroprotective agents. A further embodiment includes a screening method for identifying compounds capable of protecting central nervous system (CNS) cells from damage, comprising (a) treating dopaminergic neurons with 6-OHDA in vitro or in vivo with and without treatment with a test compound; and (b) determining the effect of the test compound on the dopaminergic neuron population, wherein a test compound capable of increasing cell survival is identified as a neuroprotective agent.

It is a further object of the present invention to provide a method for protecting CNS cells from degeneration and cell death as a result of exposure to neurotoxic substances, conditions which give rise to neurotoxic substances, and disease conditions which cause neurodegeneration, by providing a neuroprotective amount of L-ergothioneine alone, or in combination with one or more other agents that aid in protection of neuronal cells, or agents that aid in cellular proliferation and tissue regeneration. These other agents may be small synthetic organic molecules, peptides, polypeptides, nucleic acids, polynucleotides, antisense nucleotides, polyclonal or monoclonal antibodies, or other such agents that act in protecting cells of the nervous system from damage. In some embodiments of the invention, the composition may further comprise at least one ROS scavenger. Suitable ROS scavengers include coenzyme Q, vitamin E, vitamin C, pyruvate, melaton niacinamide, N-acetylcysteine, GSH, and nitrones. The other agents so described may be growth factors for neuronal cells and/or tissue. They may be agents that are ligands for particular receptors nerve cells that, upon binding, stimulate tissue regeneration or cellular proliferation. The use of combined therapy by the methods of the present invention will be dictated by the specific neuronal condition and the causative factors leading to such condition. Furthermore, L-ergothioneine may be administered along with a second agent known to enhance remyelination and/or regeneration of neurons. Methods for establishing specific dose titrations of ergothioneine and a second agent are known to those of skill in the art.

In yet another aspect of the invention there is provided a method for preventing cell death associated with acute or chronic neuronal tissue injury, the method comprising administering a therapeutically effective amount of a cocktail of antioxidants for which at least one member of the cocktail is L-ergothioneine.

Other objects and advantages will become apparent from a review of the ensuing detailed description taken in conjunction with the following illustrative drawing.

### BRIEF DESCRIPTION OF THE FIGURES

FIG. 1 shows the structure of L-ergothioneine.

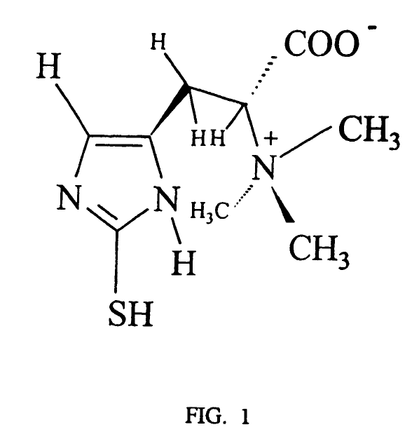

FIG. 2 are photomicrographs showing APP immunoreactivity in the right (A) and left (B) retinas of an animal that received unilateral injection of NMDA to the left eye. Note a reduction in APP immunostaining was observable in the ganglion cell layer in the NMDA-injected retina. GCL: ganglion cell layer; INL: inner nuclear layer; ONL: outer nuclear layer. Scale bar: 100 μm.

FIG. 3 are photomicrographs showing GFAP immunoreactivity in the right (A) and left (B) retinas of a rat that received unilateral injections of NMDA to the left eye. The retinal sections were counterstained lightly with cresyl violet. Note a reduction of GFAP immunostaining was observable in the astrocytes (arrow), which are located primarily on the vitreal surface of the retina in the NMDA-injected retina. Abbreviations same as in the legend to FIG. 2. Scale bar: 100 μm.

FIG. 4 are photomicrographs showing cells in the retinal ganglion cell layer in cresyl violet-stained retinal wholemounts from animals that received unilateral intravitreal injections of NMDA solution to the left eyes, and intraperitoneal injections of L-ergothioneine (A, B) or PBS (C). A and B are the right (A) and left (B) retinas from an animal treated with L-ergothioneine and C is the left retina from a rat treated with PBS. Note a significant loss of neurons in the retinal ganglion cell layer in B and C, and that the retina is less healthy in C as compared with B. Scale bar: 100 μm.

FIG. 5 is a graph showing the effect of NMDA treatment and its protection by L-ergothioneine. The neurons counted were divided into two groups with somata smaller than 6 μm, or equal to or larger than 6 μm in diameter. The great majority of neurons larger than 76 μm are retinal ganglion cells. Neurons with smaller somata are primarily non-ganglion cells or displaced amarcine cells (* =p<0.001 as compared to PBS).

FIG. 6 Protective effect of EGT on Aβ₂₅₋₃₅-induced cytotoxicity in PC12 cells A. PC12 cells were treated with the indicated amounts of Aβ₂₅₋₃₅ in the absence (closed circles) or presence of 1 mM (open circles) EGT for 36 h at 37° C. Viable cells were determined using the MTT reduction assay. EGT was added to the media 30 min prior to the Aβ₂₅₋₃₅ treatment. B. Determination of the viability of PC12 cells by LDH release after treatment with 25 μM Aβ₂₅₋₃₅ in the absence or presence of the indicated concentrations of EGT. Values are means±S.D. (n=3). There was a significant difference between the groups (* p<0.05, ** p<0.01).

FIG. 7 Protective effect of EGT on the Aβ₂₅₋₃₅-induced apoptosis. A. Effect of L-ergothioneine on terminal deoxynucleotidyl transferase-mediated dUTP nick end labelling (TUNEL). a, no treatment, b, PC12 cells exposed to 25 μM Aβ₂₅₋₃₅ for 36 h; c, Aβ₂₅₋₃₅ (25 μM)+EGT (0.5 mM); d, Aβ₂₅₋₃₅ (25 μM)+EGT (1 mM). There was a significant difference between the groups (* p<0.05, ** p<0.01). B. Effect of EGT on the mitochondrial membrane potential. ΔΨm was assessed with the TMRE fluorescence as described in Materials and Methods below. a, no treatment; b, PC12 cells exposed to 25 μM Aβ₂₅₋₃₅ for 36 h; c, Aβ₂₅₋₃₅ (25 μM)+EGT (0.5 mM); d, Aβ₂₅₋₃₅ (25 μM)+EGT (1 mM).

FIG. 8 Effect of EGT on the Aβ₂₅₋₃₅-induced apoptotic signaling pathway. PC12 cells were incubated with 25 μM Aβ₂₅₋₃₅ for 36 h in the presence or absence of indicated concentrations of EGT and harvested for Western blot analysis. A. EGT attenuated Aβ₂₅₋₃₅-induced cleavage of PARP as determined by using anti-PARP antibody (upper panel). Actin levels were measured for the confirmation of equal amount of protein loading (lower panel). B. Effect of EGT on the levels of Bax (upper panel) and Bcl-X_{L} (lower panel). There was a significant difference between the groups (* p<0.05, ** p<0.01).

FIG. 9 Effect of EGT on the Aβ₂₅₋₃₅-induced peroxynitrite formation and lipid peroxidation A. Left panel: Representative confocal micrographs of DHR-derived fluorescence in PC12 cells exposed to Aβ₂₅₋₃₅ alone or in combination with EGT. Illumination and image acquisition conditions are given in the Materials and Methods. Right panel: Quantitive analysis of the DHR fluorescence intensity after treatment with Aβ₂₅₋₃₅ in the absence or presence of EGT. B. Effect of EGT on lipid peroxidation in PC12 cells. PC12 cells were exposed to 25 μM Aβ₂₅₋₃₅ for 36 h in the presence or absence of indicated concentrations of EGT. Lipid peroxidation was determined by measuring the levels of malonedialdehyde (MDA) formed. The average amount of MDA in untreated control cells was 2.01 nmole/mg protein. There was a significant difference between the groups (* p<0.05, ** p<0.01).

FIG. 10 Effect of EGT on cell death induced by the NO releasing compound, SNP (A) and by peroxynitrite generating SIN-1 (B) EGT exerted a concentration-dependent protection of SIN-1-mediated cell death but not the SNP-caused cell death. Viable cells were determined using the MTT reduction assay. Values are means±S.D. (n=3). There was a significant difference between the groups (* p<0.05, ** p<0.01).

FIG. 11A. The inhibitory effect of EGT on Aβ₂₅₋₃₅-induced NF-κB DNA binding active Nuclear extracts prepared from PC12 cells treated with Aβ₂₅₋₃₅ for 1 h in the absence or presence varying concentrations of EGT were subjected to EMSA. Lane 1, DMSO control; lane 2, Aβ₂₅₋₃₅ (25μM) alone; lane 3, Aβ₂₅₋₃₅ (25 μM)+EGT (0.5 mM); lane 4, Aβ₂₅₋₃₅ (25 μM)+EGT (1 mM). B. 7 inhibitory effect of EGT on Aβ₂₅₋₃₅-induced nuclear translocation of p65. PC12 cells treated with Aβ₂ for 1 h were fixed with 10% neutral buffered-formalin solution then incubated with anti-p65 antibody immunocytochemistry as described in Materials and Methods.

FIG. 12 A proposed molecular mechanism for the protective effect of EGT against Aβ-induced nitrosative cell death.

### DETAILED DESCRIPTION OF THE INVENTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a screening assay” include one or more assays, reference to “the formulation” or “the method” includes one or more formulations, methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Definitions

An “antibody” is any immunoglobulin, including antibodies and fragments thereof, such as Fab or F(ab′)₂ that binds a specific epitope. The term encompasses, inter alia, polyclonal, monoclonal, and chimeric antibodies, the last mentioned described in further detail in U.S. Pat. Nos. 4,816,39.7 and 4,816,567. The term also encompasses human and/or humanized antibodies. An antibody preparation is reactive for a particular antigen when at least a portion of the individual immunoglobulin molecules in the preparation recognize (i.e., bind to) the antigen. An antibody preparation is non-reactive for an antigen when binding of the individual immunoglobulin molecules in the preparation to the antigen is not detectable by commonly used methods.

The term “substantially pure,” when referring to a polypeptide, means a polypeptide that is at least 60%, by weight, free from the proteins and naturally-occurring organic molecules with which it is naturally associated. A substantially pure composition of L-ergothioneine is at least 75%, more preferably at least 90%, and most preferably at least 99%, by weight, L-ergothioneine.

L-Ergothioneine can be obtained, for example, by chemical synthesis or by isolation from natural sources. Purity can be measured by any appropriate method, e.g., column chromatography, polyacrylamide gel electrophoresis, HPLC analysis, and chiral methods. Chiral purity is important and can be assayed by known methods, including chiral chromatography or optical rotation.

“Treatment” refers to the administration of medicine or the performance of medical procedures with respect to a patient, for either prophylaxis (prevention) or to cure the infirmity or malady in the instance where the patient is afflicted.

A “therapeutically effective amount” or “efficacious amount” is an amount of a reagent sufficient to achieve the desired treatment effect. A “neuroprotectively effective amount” is an amount of L-ergothioneine that is sufficient to protect against neuronal loss. Amounts effective for this use will depend on the severity of the condition, the general state of the patient, the route of administration, and other factors known to those skilled in the art. For example, the doses of L-ergothioneine or other compounds identified by the methods of the present invention, that protect against neuronal cell death, could range from 10 mg to 10 grams daily, depending on the severity of disease and specifics of treatment, and whether the compound is administered in combination with another compound used to promote cell proliferation or tissue regeneration, cell survival or outgrowth of neuronal processes.

The term “trophic effects” means that the “neurotrophic factor” of the present invention has selective effects on specific neural elements that contribute to the survival, growth, maturation and regeneration of neurons present in the nervous tissue.

“Mucosal” refers to the tissues in the body that secrete mucous; thus encompassing the oral cavity (nose, throat, and mouth), the digestive tract (including the intestines), as well as the rectum and vagina.

“Transmucosal” refers to the passage of materials across or through the mucosal membranes.

“Sublingual” refers to the area under the tongue.

“Sublingual delivery” refers to the systemic delivery of drugs or other agents through the mucosal membranes lining the floor of the mouth.

“Buccal” refers to the cheek area in the mouth.

“Buccal delivery” refers to administration of drugs or other agents through the mucosal membranes lining the cheeks (buccal mucosa).

General Aspects of the Invention

Finding a means of protecting neuronal cells from the effects of toxic substances is of obvious medical importance. It is known that many substances present in the surrounding environment of a cell can influence cell death or survival. In particular, cell death may be attributed to the presence of substances such glutamate, complement, tumor necrosis factor-α, gamma interferon or other cytokines, as well as reactive oxygen species (ROS) or reactive nitrogen species (RNS). These toxic compounds, as well as others, have been associated with a large variety of conditions in which cells die and such cell death causes severe clinical consequences. Such is the case in many conditions that affect the nervous system. Thus, it is a matter of significant importance to identify therapeutic compounds or combinations thereof that would prevent such cell death and which might be applicable in a clinical setting. Furthermore, identifying agents that act as neuroprotectants in a variety of situations whereby such neuroprotectant activity is desirable, such as in acute or chronic nerve injuries, for example, traumatic brain injury or spinal cord injury, or in other diseases or conditions affecting the central nervous system is of utmost importance. In addition, the identification of agents that act as neuroprotectants, and which show increased efficacy when combined with other agents that enhance or promote cell division, cell survival and outgrowth of neuronal processes will find important use in many clinical applications, ranging from treatment of chronic degenerative disorders and acute injury. For example, treatment of multiple sclerosis patients during an acute relapse could conceivably reduce the destruction of oligodendrocytes occurring in the lesions of these patients. Yet further, the use of the agents of the present invention could be extremely beneficial when used alone or in combination with one or more additional treatment regimens in conditions such as stroke or Alzheimer's disease or Parkinson's disease where ongoing neuronal cell death leads to further loss of function in patients having these disorders.

Furthermore, it is generally recognized that many disease processes are attributed to the presence of elevated levels of free radicals and reactive oxygen species (ROS) and reactive nitrogen species (RNS), such as superoxide, hydrogen peroxide, singlet oxygen, peroxynitrite, hydroxyl radicals, hypochlorous acid (and other hypohalous acids) and nitric oxide. In the eye, cataract, macular degeneration and degenerative retinal damage are attributed to ROS. Among other organs and their ROS-related diseases include: lung cancer induced by tobacco combustion products and asbestos; accelerated aging and its manifestations, including skin damage; atherosclerosis; ischemia and reperfusion injury, diseases of the nervous system such as Parkinson disease, Alzheimer disease, muscular dystrophy, multiple sclerosis; lung diseases including emphysema and bronchopulmonary dysphasia; iron overload diseases such as hemochromatosis and thalassemia; pancreatitis; renal diseases including autoimmune nephrotic syndrome and heavy metal-induced nephrotoxicity; and radiation injuries. Certain anti-neoplastic drugs such as adriamycin and bleomycin induce severe oxidative damage, especially to the heart, limiting the patient's exposure to the drug. Redox-active metals such as iron induce oxidative damage to tissues; industrial chemicals and ethanol, by exposure and consumption, induce an array of oxidative damage-related injuries, such as cardiomyopathy and liver damage. Airborne industrial and petrochemical-based pollutants, such as ozone, nitric oxide, radioactive particulates, and halogenated hydrocarbons, induce oxidative damage to the lungs, gastrointestinal tract, and other organs. Radiation poisoning from industrial sources, including leaks from nuclear reactors and exposure to nuclear weapons, are other sources of radiation and radical damage. Other routes of exposure may occur from living or working in proximity to sources of electromagnetic radiation, such as electric power plants and high-voltage power lines, x-ray machines, particle accelerators, radar antennas, radio antennas, and the like, as well as using electronic products and gadgets which emit electromagnetic radiation such as cellular telephones, and television and computer monitors.

The present invention provides methods of specifically protecting neuronal cells of the mammalian body from damage attributed to neurotoxic substances by the application or administration of a composition comprising L-ergothioneine and a suitable carrier. The neurotoxic substances may be agents such as glutamate or glutamate analogs, or they may be anticancer agents or other agents useful in treating conditions other than nervous system disorders. L-ergothioneine may protect against neural damage resulting from exposure to cytokines such as, for example, tumor necrosis factor alpha or gamma interferon, or one or more free radicals and oxidants such as, for example, singlet oxygen, hydroxyl radicals, peroxyl radicals, peroxynitrite, hydrogen peroxide, nitric oxide, hypochlorous acid (and other hypohalous acids) and/or metalloenzymes. Other neurotoxic effects for which L-ergothioneine may be beneficial may result from radiation therapy or the release of free radicals from cells following an injury to neural tissue, such as a brain trauma, a stroke, or a spinal cord injury. In another embodiment, L-ergothioneine may protect against neural damage caused by the presence of a neurodegenerative disease, such as for example, Alzheimer's disease, multiple sclerosis, Down's syndrome, amyotropic lateral sclerosis, Parkinson's disease, macular degeneration, HIV/AIDS and optic neuropathies and retinopathies.

The multifunctional nature of L-ergothioneine makes it a candidate for investigation of its therapeutic use in conditions such as Parkinson's Disease (PD). One aspect of the instant invention is based-in part on the discovery of neuroprotective properties observed for L-ergothioneine in the unilateral 6-hydroxydopamine (6-OHDA) lesion rat model of PD. The integrity, e.g., number of dopaminergic cell bodies in the substantia nigra estimated by immuno-staining for tyrosine hydroxylase (TH) and functionality of striatal dopamine levels estimated by HPLC of the nigro-striatal dopaminergic system were investigated. TH is the rate limiting enzyme in dopamine synthesis.

Furthermore, the same multifunctional properties of L-ergothioneine that make it a candidate use in Parkinson's disease also make it applicable for use in the treatment of Alzheimer's disease. As noted previously, Alzheimer's disease (AD) is a chronic neurodegenerative disorder and is characterized by the accumulation of neurofibrillary tangles and senile plaques, and a widespread progressive degeneration of neurons in the brain. Senile plaques are rich in amyloid precursor protein (APP) that is encoded by the APP gene located on chromosome 21. A commonly accepted hypothesis underlying pathogenesis of AD is that abnormal proteolytic cleavage of APP leads to an excess extracellular accumulation of beta-amyloid (Aβ) peptide that has been shown to be toxic to neurons (Selkoe et al., (1996), J. Biol. Chem. 271:487-498; Quinn et al.; (2001), Exp. Neurol. 168:203-212; Mattson, et al., (1997), Alzheimer's Dis. Rev. 12:1-14; Fakuyama et al., (1994), Brain Res. 667:269-272).

Injection of neurotoxins, for example, the aggregated β-amyloid peptides, into the vitreal body of rats results in severe degeneration of neurons in the retina. These effects can be ameliorated to some extent by co-treatment with a single injection of the antioxidant vitamin E (Jen et al. (1998) Nature 392:140-141). This suggests that oxidative stress in vivo plays a role in causing degeneration of neurons in the retina. The mammalian retina is an integral part of the central nervous system but it is peripherally located and therefore highly accessible experimentally. The retina has an organized structure with biochemically and structurally defined glial and neuronal populations. Furthermore, it is a closed system and provides an ideal way of assessing the effectiveness of specific chemical compounds that are known to be neuroprotective or neurotoxic.

As shown in the instant application, injection of aggregated β-amyloid peptides, Aβ₂₅₋₃₅ (Aβ) into the vitreal body of rats resulted in severe degeneration of neurons in the retina. Furthermore, data is presented in the present application which supports the beneficial effects of L-ergothioneine and suggests its potential for use as stand-alone therapy in Alzheimer's disease, or its potential for use in combination with other agents or regimens in attenuation of the progression of Alzheimer's disease.

The method of the invention is useful with any mammal of interest. In a preferred embodiment, the mammal is a human being. A further embodiment would be for veterinary use in the treatment of domestic and non-domestic animals having suffered a traumatic injury.

In further embodiments, L-ergothioneine is administered as a dietary supplement in an amount effective to provide protection from neurotoxic compounds. In more specific embodiments, the dietary supplement is in the form of an oral capsule or tablet or a liquid suspension. Other embodiments include administration of L-ergothioneine in a form suitable for sublingual or buccal delivery. Further embodiments include delivery of L-ergothioneine in a suppository form. Yet further embodiments include formulations of L-ergothioneine suitable for intrathecal, intraventricular or intracranial delivery. The specific embodiment utilized is dictated by the condition of the patient to be treated. In certain conditions, such as following a stroke, the patient's ability to swallow is compromised, thus there is a need to deliver L-ergothioneine or other active compounds identified by the methods of the present invention via a route that does not involve swallowing.

In a further embodiment, L-ergothioneine is administered directly to the site of injury in an amount effective to inhibit the damage attributed to the release of free radicals and oxidants from injured cells and damaged tissue. In the case of a traumatic injury, such as a brain or an acute or chronic spinal cord injury, L-ergothioneine may be delivered intrathecally, intracranially or intraventricularly.

In a related second aspect, the invention features a method of protecting a mammalian neural cell from neurodegeneration, comprising administering a therapeutically effective amount of L-ergothioneine to a mammal in need thereof. One specific embodiment includes a method of protecting a mammalian neural cell from neurodegeneration by administration of a pharmaceutical composition comprising L-ergothioneine and a pharmaceutically acceptable carrier. Such pharmaceutical compositions may be designed for oral delivery, intravenous delivery, intramuscular delivery, subcutaneous delivery, intrathecal delivery or intraventricular delivery. Certain embodiments may include specific carrier molecules that aid in ergothioneine crossing the blood brain barrier.

It is a further object of the present invention to provide a method for protecting CNS cells from degeneration and cell death as a result of exposure to neurotoxic substances, conditions which give rise to neurotoxic substances, and disease conditions which cause neurodegeneration, by administering a neuroprotective amount of L-ergothioneine alone, or in combination with one or more other agents that aid in protection of neuronal cells, or agents that aid in cellular proliferation and tissue regeneration. These other agents may be small synthetic organic compounds, proteins, peptides, polypeptides, nucleic acids, polynucleotides, antisense oligonucleotides, polyclonal or monoclonal antibodies, or other such agents that act in protecting cells of the nervous system from damage or that promote cell survival and/or promote tissue regeneration and/or remyelination.

In some embodiments of the invention, the composition may further comprise at least one ROS scavenger. Suitable ROS scavengers include coenzyme Q, vitamin E, vitamin C, pyruvate, melatonin, niacinamide, N-acetylcysteine, GSH, and nitrones. The other agents so described may be growth factors for neuronal cells and/or tissue. They may be agents that are ligands for particular receptors on nerve cells that, upon binding, stimulate tissue regeneration or cellular proliferation.

The use of combined therapy by the methods of the present invention will be dictated by the specific neuronal condition and the causative factors leading to such condition. Furthermore, L-ergothioneine may be administered along with a second agent known to enhance remyelination and regeneration of neurons. Methods for establishing specific dose titrations of L-ergothioneine and a second agent are known to those of skill in the art.

In yet another aspect of the invention there is provided a method for preventing cell death associated with acute or chronic neuronal tissue injury, the method comprising administering a therapeutically effective amount of a cocktail of antioxidants for which at least one member of the cocktail is L-ergothioneine. The second antioxidant may be, for example, vitamin C or vitamin E.

The proteins useful in combination therapy with L-ergothioneine may be neurotrophic factors. Neurotrophic factors are a class of molecules that have been initially identified as participants in the development of vertebrate nervous systems by facilitating the interaction of neurons with their target cells. It has been observed that competition among neurons for such target cells takes place and that only those neurons that achieve such interaction will survive (Leibrock et al., 1989, Nature, 341:149; Hohn et al., 1990, Nature, 344:339). Accordingly, such neurotrophic factors promote the survival and functional activity of nerve or glial cells. Evidence also exists to suggest that neurotrophic factors will be useful as treatments to prevent nerve or glial cell death or malfunction resulting from the conditions enumerated above (Appel, 1981, Ann. Neurology, 10:499; U.S. Pat. Nos. 4,699,875 and 4,701,407 to Appel; U.S. Pat. No. 4,923,696 to Appel et al.).

The best characterized of such neurotrophic factors is nerve growth factor (NGF). NGF has been demonstrated to be a neurotrophic factor for the forebrain cholinergic nerve cells that die during Alzheimer's disease and with increasing age. The loss of these nerve cells is generally considered responsible for many of the cognitive deficits associated with Alzheimer's disease and with advanced age.

Experiments in animals demonstrate that NGF prevents the death of forebrain cholinergic nerve cells after traumatic injury and that NGF can reverse cognitive losses that occur with aging (Hefti & Weiner, 1986, Ann. Neurology, 20:275; Fischer et al., 1987, Nature, 329:65). These results suggest the potential clinical utility in humans of this neurotrophic factor in the treatment of cognitive losses resulting from the death of forebrain cholinergic nerve cells through disease, injury or aging.

Other neurotrophic factors have been isolated and characterized, among them brain-derived neurotrophic factor (BDNF) (Leibrock et al., supra.); a variant named hippocampus-derived neurotrophic factor (HDNF) (Ernfors et al., 1990, Proc. Natl. Acad. Sci. USA, 87:5454); neurotrophin-3 (NT-3) (Hohn et al., supra.; Maisonpierre et al., 1990, Science, 247:1446; Rosenthal et al., 1990, Neuron, 4:767); and Ciliary Neurotrophic Factor (CNTF) (Kishimoto, T., Taga, T., and Akira, S. Cell, 76:252-262, 1994; Stahl, N. and Yancopoulos, G. D., Cell 74:587-590, 1994). All of the foregoing are incorporated herein by reference.

Other agents that may be used in conjunction with L-ergothioneine or with the novel agents identified by the methods of the present invention may be ligands that stimulate cell proliferation and survival. For example, these ligands may include those that bind to and activate receptor protein kinases and receptors associated with tyrosine kinases (van der Geer, P., Hunter, T. and Lindberg, R. A., Ann. Rev. Cell Biol. 10:251-337, 1994). They may be agonist ligands for integrins (Chothis, C. and Jonnes, E. Y., Ann. Rev. Biochem. 66:823-862, 1997). Such molecules may include laminin, which is known in the art to promote neurite outgrowth (Bates, C. A. and Meyer, R. L., Dev. Biol. 181:91-101, 1997). Other molecules may be derived from the immunoglobulin superfamily (Walsh, F. S. and Doherty, P. Ann. Rev. Cell Dev. Biol. 13:425-456, 1997). It is also possible to develop molecules that act as receptor mimics that exhibit the same properties as the native agonist ligand. All of the above could be suitable for use in conjunction with L-ergothioneine or with the novel neuroprotective agents identified by the methods of the present invention.

The experiments presented below show that dietary L-ergothioneine was effective in protecting retinal neurons, and the neuroprotective effect was more pronounced for the ganglion cell population as compared with the non-ganglion cell population. There was a slight reduction in cell density and/or degeneration of the total neuronal population in the un-injected retina, suggesting a non-specific and systemic effect of unilateral injection of neurotoxic chemical compounds.

However, the experiments demonstrate that intraperitoneal injection of ergothioneine protected neurons from experimentally induced degeneration or loss due to NMDA toxicity, thus also demonstrating its ability to cross the blood brain barrier. Further, the retinal system in mammals is shown to be a useful in vivo experimental model for studying factors that affect neuronal development, function, or survival.

Pharmaceutical Compositions and Methods of Administration

The present invention also provides pharmaceutical compositions used in the method of the invention. Such compositions comprise a therapeutically effective amount of L-ergothioneine, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Administration of L-ergothioneine to the site of injury, the target cells, tissues, or organs, may be by way of oral administration as a pill or capsule or a liquid formulation or suspension. It may be administered via the transmucosal, sublingual, nasal, rectal or transdermal route. Parenteral administration may also be via intravenous injection, or intraarterial, intramuscular, intradermal, subcutaneous, intraperitoneal, intraventricular, intrathecal and intracranial administration. For example, the composition of the present invention may be infused directly into a tissue or organ that had undergone an infarct, such as the brain or heart following a stroke or heart attack, in order to protect mitochondria in the cells of the ischemic penumbra, those outside of the immediate infarct zone which are not killed during the cessation of blood flow but undergo extensive ROS-mediated damage when blood flow is restored. Due to the nature of the neurological diseases or conditions for which the present invention is being considered, the route of administration may also involve delivery via suppositories. This is especially true in conditions such as stroke whereby the ability of the patient to swallow is compromised.

L-Ergothioneine may be provided as a liposome formulation. Liposome delivery has been utilized as a pharmaceutical delivery system for other compounds for a variety of applications. See, for example Langer (1990) Science 249:1527-1533; Treat et al. (1989) in Liposomes in the Therapy of Infectious Disease and Cancer, Lopez-Berestein and Fidler (eds.), Liss: New York, pp. 353-365 (1989). Many suitable liposome formulations are known to the skilled artisan, and may be employed for the purposes of the present invention. For example, see: U.S. Pat. No. 5,190,762.

In a further aspect, L-ergothioneine liposomes can cross the blood-brain barrier, which would allow for intravenous or oral administration. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier; and the like. In another embodiment, the molecule can be administered intracranially or, more preferably, intraventricularly. In yet another embodiment, L-ergothioneine can be administered in a liposome targeted to the blood-brain barrier.

Transdermal delivery of L-ergothioneine, either as a liposome formulation or free L-ergothioneine, is also contemplated. Various and numerous methods are known in the art for transdermal administration of a drug, e.g., via a transdermal patch. It can be readily appreciated that a transdermal route of administration may be enhanced by use of a dermal penetration enhancer.

Controlled release oral formulations may be desirable when practicing the neuroprotective method of the invention. The drug may be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Pulmonary delivery of L-ergothioneine may be used for treatment as well. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

Ophthalmic and nasal delivery of L-ergothioneine may be used in the method of the invention. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextrins. For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

The compositions and formulations of the present invention are suited for the transmucosal delivery of L-ergothioneine. In particular, the compositions and formulations are particularly suited for sublingual, buccal or rectal delivery of agents that are sensitive to degradation by proteases present in gastric or other bodily fluids having enhanced enzymatic activity. Moreover, transmucosal delivery systems can be used for agents that have low oral bioavailability. The compositions of the instant invention comprise L-ergothioneine dissolved or dispersed in a carrier that comprises a solvent, an optional hydrogel, and an agent that enhances transport across the mucosal membrane. The solvent may be a non-toxic alcohol known in the art as being useful in such formulations of the present invention and may include, but not be limited to ethanol, isopropanol, stearyl alcohol, propylene glycol, polyethylene glycol, and other solvents having similar dissolution characteristics. Other such solvents known in the art can be found in The Handbook of Pharmaceutical Excipients, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Any transmucosal preparation suitable for administering the components of the present invention or a pharmaceutically acceptable salt thereof can be used. Particularly, the mixture is any preparation usable in oral, nasal, or rectal cavities that can be formulated using conventional techniques well known in the art. Preferred preparations are those usable in oral, nasal or rectal cavities. For example, the preparation can be a buccal tablet, a sublingual tablet, and the like preparation that dissolve or disintegrate, delivering drug into the mouth of the patient. A spray or drops can be used to deliver the drug to the nasal cavity. A suppository can be used to deliver the mixture to the rectal mucosa. The preparation may or may not deliver the drug in a sustained release fashion.

A specific embodiment for delivery of the components of the present invention is a mucoadhesive preparation. A mucoadhesive preparation is a preparation which upon contact with intact mucous membrane adheres to said mucous membrane for a sufficient time period to induce the desired therapeutic or nutritional effect. The preparation can be a semisolid composition as described for example, in WO 96/09829. It can be a tablet, a powder, a gel or film comprising a mucoadhesive matrix as described for example, in WO 96/30013. The mixture can be prepared as a syrup that adheres to the mucous membrane.

Suitable mucoadhesives include those well known in the art such as polyacrylic acids, preferably having the molecular weight between from about 450,000 to about 4,000,000, for example, Carbopol™934P; sodium carboxymethylcellulose (NaCMC), hydroxypropylmethylcellulose (HPMC), or for example, Methocel.™. K100, and hydroxypropylcellulose.

The delivery of the components of the present invention can also be accomplished using a bandage, patch, device and any similar devide that contains the components of the present invention and adheres to a mucosal surface. Suitable transmucosal patches are described for example in WO 93/23011, and in U.S. Pat. No. 5,122,127, both of which are hereby incorporated by reference. The patch is designed to deliver the mixture in proportion to the size of the drug/mucosa interface. Accordingly, delivery rates can be adjusted by altering the size of the contact area. The patch that may be best suited for delivery of the components of the present invention may comprise a backing, such backing acting as a barrier for loss of the components of the present invention from the patch. The backing can be any of the conventional materials used in such patches including, but not limited to, polyethylene, ethyl-vinyl acetate copolymer, polyurethane land the like. In a patch that is made of a matrix that is not itself a mucoadhesive, the matrix containing the components of the present invention can be coupled with a mucoadhesive component (such as a mucoadhesive described above) so that the patch may be retained on the mucosal surface. Such patches-can be prepared by methods well known to those skilled in the art.

Preparations usable according to the invention can contain other ingredients, such as fillers, lubricants, disintegrants, solubilizing vehicles, flavours, dyes and the like. It may be desirable in some instances to incorporate a mucous membrane penetration enhancer into the preparation. Suitable penetration enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, poly xyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

The administration of the compounds of the present invention can be alone, or in combination with other compounds effective at treating the various medical conditions contemplated by the present invention. Also, the compositions and formulations of the present invention, may be administered with a variety of analgesics, anesthetics, or anxiolytics to increase patient comfort during treatment.

The compositions of the invention described herein may be in the form of a liquid. The liquid may be delivered as a spray, a paste, a gel, or a liquid drop. The desired consistency is achieved by adding in one or more hydrogels, substances that absorb water to create materials with various viscosities. Hydrogels that are suitable for use are well known in the art. See, for example, Handbook of Pharmaceutical Excipients, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Suitable hydrogels for use in the compositions include, but are not limited to, hydroxypropyl cellulose, hydroxypropyl methyl cellulose, sodium carboxymethyl cellulose and polyacrylic acid. Preferred hydrogels are cellulose ethers such as hydroxyalkylcellulose. The concentration of the hydroxycellulose used in the composition is dependent upon the particular viscosity grade used and the viscosity desired in the final product. Numerous other hydrogels are known in the art and the skilled artisan could easily ascertain the most appropriate hydrogel suitable for use in the instant invention.

The mucosal transport enhancing agents useful with the present invention facilitate the transport of the agents in the claimed invention across the mucosal membrane and into the blood stream of the patient. The mucosal transport enhancing agents are also known in the art, as noted in U.S. Pat. No. 5,284,657, incorporated herein by reference. These agents may be selected from the group of essential or volatile oils, or from non-toxic, pharmaceutically acceptable inorganic and organic acids. The essential or volatile oils may include peppermint oil, spearmint oil, menthol, eucalyptus oil, cinnamon oil, ginger oil, fennel oil, dill oil, and the like. The suitable inorganic or organic acids useful for the instant invention include but are not limited to hydrochloric acid, phosphoric acid, aromatic and aliphatic monocarboxylic or dicarboxylic acids such as acetic acid, citric acid, lactic acid, oleic acid, linoleic acid, palmitic acid, benzoic acid, salicylic acid, and other acids having similar characteristics. The term “aromatic” acid means any acid having a 6-membered ring system characteristic of benzene, whereas the term “aliphatic” acid refers to any acid having a straight chain or branched chain saturated or unsaturated hydrocarbon backbone.

Other suitable transport enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

When the compositions and formulations of the instant invention are to be administered to the oral mucosa, the preferred pH should be in the range of pH 3 to about pH 7, with any necessary adjustments made using pharmaceutically acceptable, non-toxic buffer systems generally known in the art.

For topical delivery, a solution of L-ergothioneine in water, buffered aqueous solution or other pharmaceutically-acceptable carrier, or in a hydrogel lotion or cream, comprising an emulsion of an aqueous and hydrophobic phase, at a concentration of between 50 μM and 5 mM, is used. A preferred concentration is about 1 mM. To this may be added ascorbic acid or its salts, or other ingredients, or a combination of these, to make a cosmetically-acceptable formulation. Metals should be kept to a minimum. It may be preferably formulated by encapsulation into a liposome for oral, parenteral, or, preferably, topical administration.

The invention provides methods of treatment comprising administering to a subject a neuroprotectively effective amount of L-ergothioneine. In one embodiment, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In one specific embodiment, a non-human mammal is the subject. In another specific embodiment, a human mammal is the subject.

The amount of L-ergothioneine which is optimal in protecting neuronal cells from damage can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from in vitro or animal model test systems.

Treatment Group

A subject in whom administration of L-ergothioneine is an effective therapeutic regiment for neuroprotection is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The protection of neuronal cells from damage from neurotoxic substances or conditions should be considered when possible prior to exposure to such neurotoxic substances and conditions. Exposoure to neurotoxic substances and conditions may be considered in the presence of diseases and disorders known to result in neurodegeneration, e.g., in the presence of Alzheimer's disease. Further, exposure to neurotoxins, pollutants, radiation such as solar, electromagnetic or nuclear, and to pharmaceuticals known to, generate reactive oxygen species and other radicals, are recognized as potentially harmful to cells of the CNS. The neuroprotective method of the invention may be used prior to exposure to neurotoxic substances or conditions to reduce or prevent neuronal damage. Furthermore, the administration of L-ergothioneine may be given at the time of or after the injury or exposure to the neurotoxic substance, alone, or in combination with other agents known to be neuroprotective or known to be beneficial for stimulating repair of, or regeneration of, neural tissue, or to aid in neuronal cell proliferation, or beneficial to remyelination.

Screening for Neuroprotectant Agents

In a third aspect, the invention features a screening method for identifying compounds capable of protecting central nervous system cells from damage, comprising (a) exposing (treating) retinal neurons to neurotoxic agents with and without treatment with test compounds; and (b) determining the effect of the test compounds on retinal neuron populations, wherein test compounds capable of increasing neuronal integrity or preserving neuronal cell numbers are identified as neuroprotective agents. A further embodiment includes a screening method for identifying compounds capable of protecting central nervous system (CNS) cells from damage, comprising (a) treating dopaminergic neurons with 6-OHDA in vitro or in vivo with and without treatment with a test compound; and (b) determining the effect of the test compound on the dopaminergic neuron population, wherein a test compound capable of increasing cell survival is identified as a neuroprotective agent. A yet further embodiment would be screening for novel compounds capable of protecting central nervous system cells from damage using the methods described above and using L-ergothioneine as a standard or positive control for efficacy in the assay.

Specific Neuroprotective Effects of L-Ergothioneine

Intravitreal Injection of NMDA and Neuroprotection by L-Ergothioneine

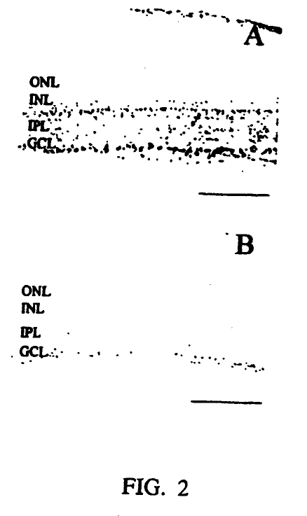

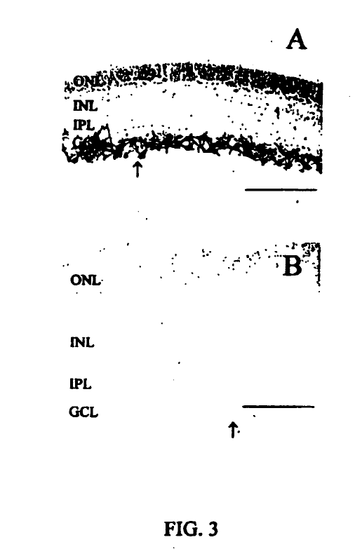

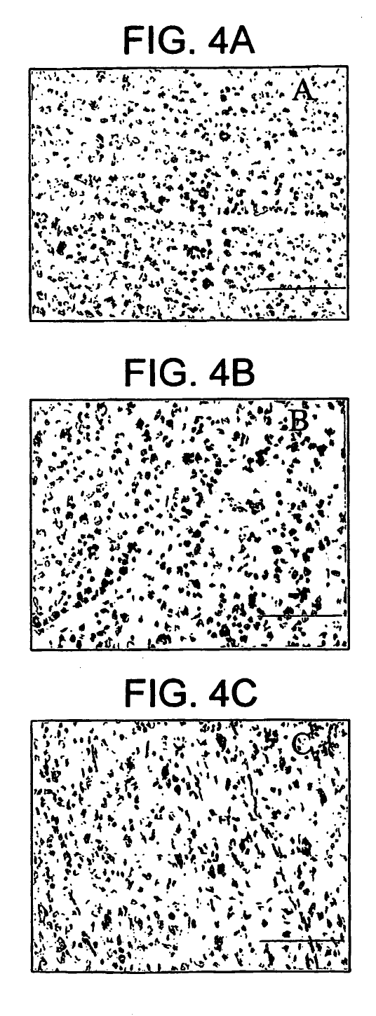

In accordance with this aspect of the invention, rats injected intravitreally with NMDA without administration of L-ergothioneine, demonstrated an apparent reduction in immunostaining for amyloid precursor protein (APP) in ganglion cells (FIG. 2). Similarly, a reduction in immunoreactivity of glial fibrillary acidic protein (GFAP) was also detected in astrocytes that were located primarily on the vitreal surface of the retina in NMDA-injected retinas (FIG. 3). In histological sections stained for cresyl violet, the retinal tissue obtained from rats injected with NMDA for 24 hours appeared to be less healthy, degenerative or necrotic, as compared with the normal or uninjected retinas (FIG. 4).

In normal retinas, the total average cell density is 6394 cells/mm². Of these, 61% are non-ganglion cells and 39% are considered as ganglion cells on the basis of their somal diameter. These figures are in line with previous studies, see for example, Perry (1981) supra, showing that more than half of the entire population of neurons in the ganglion cell layer are displaced amacrine cells with small somata as compared with the ganglion cells.

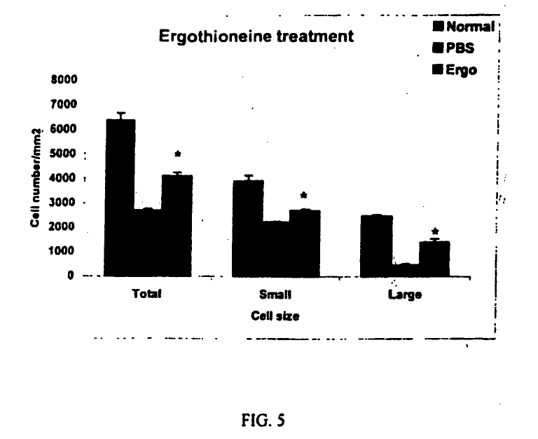

In animals that received intravitreal injection of NMDA and were treated with PBS, there was a 58% reduction in total cell numbers in the retina. This reduction was particularly apparent in the larger cells with an 81% loss of ganglion cells and a 43% reduction in non-ganglion cells. In contrast, there was a loss of only 15% of ganglion cells and 8% of non-ganglion cells in the uninjected retinas (FIGS. 4-5). In L-ergothioneine treated animals, there was a loss of 44% of ganglion cells and 31% of the small or non-ganglion cells. The uninjected control eyes from these animals showed a loss of 7% and 4% of these populations (FIG. 5).

NMDA is excitotoxic to neurons. In order to ascertain that intravitreal injection of NMDA actually led to a loss and not atrophy of neurons in the retina, cell count and size measurement were performed in retinal wholemounts 6 weeks after injection of NMDA, a time point greater than reported in earlier studies (Laabich et al. (2000) Mol. Brain Res. 85:32-40), and the results are in accord with previous studies showing a neurotoxic effect of NMDA on retinal neurons (Kido et al. (2000) Brain Res. 884:59-67; Laabich et al. (2000) supra).

The present invention provides evidence of an in vivo effect of NMDA in causing significant degeneration and loss of both ganglion and displaced amacrine cell populations in the ganglion cell layer. The cytotoxic effect of NMDA appears to be more severe in the ganglion cell populations that are known to be primarily glutamatergic (Fletcher et al. (2000) J. Comp. Neurol. 420:98-112). This is consistent with our observations of a reduction of APP in the ganglion cells. The fact that there was a reduction in displaced amacrine cells which are mainly non-glutamatergic suggests that the cytotoxic effects of NMDA may not be specific or limited to the ganglion layer cell populations. This may be in keeping with the suggestion that a subpopulation of amacrine/displaced amacrine cells may express NMDA receptors,. and thus may be vulnerable to excitotoxicity (Fletcher et al. (2000) supra).

However, the reduction of GFAP immunoreactivity in astrocytes after NMDA injection implies that there may also be an indirect detrimental effect of NMDA treatment on non-glutamatergic neurons or neurons that do not express NMDA receptors via glial cell dysfunction. Indeed, retinal glial cells are known to play an important role in normal function and survival of retinal neurons. Dysfunction of these cells may be the precipitating factor of neuronal degeneration in retinas challenged by insults of a different nature, e.g., cytotoxic β-amyloid peptides (Jen et al. (1998) supra; Aruoma et al. (1999) supra).

The observed reduction of small cell populations with somal diameter less than 6 μm in addition to a reduction of the larger ganglion cells 6 weeks after intravitreal injection of NMDA indicates that there is actual loss of the neuronal population in the ganglion cell layer rather than of the larger cells. This loss is most likely to be permanent and rules out the possibility of reversible degenerative changes as indicated by shrinkage of somata.

Effects of L-Ergothioneine on Aβ Cytotoxicity of P12 Cells

Beta-Amyloid peptide is the major component of senile plaques and considered to have a causal role in the development and progression of Alzheimer's disease (AD). In the present application, results are shown which demonstrate a positive effect of L-ergothioneine on prevention of Aβ-induced-oxidative cell death. Rat pheochromocytoma (PC12) cells were used for testing the effects of L-ergothioneine on protection from cell death following exposure to Aβ. The PC12 cells are a well defined in vitro model for studies of neuronal cell death and differentiation (Fujita et al. (1989), Environ. Health Perspect. 80:127-142; Leclerc et al. (1995), Neurosci. Lett. 201:103-106). These cells retain phenotypic characteristics of adrenal chromaffin cells and sympathetic neurons (Green et al. (1976), Proc. Natl. Acad. Sci. USA 73:2424-2428). Cells treated with Aβ underwent apoptotic death as determined by positive in situ terminal end-labelling (TUNEL staining), decreased mitochondrial membrane potential (ΔΨm), an increased ratio of proapoptotic Bax to antiapoptotic BCl-X_{L} and the cleavage of poly(ADP-ribose) polymerase. Treatment with L-ergothioneine attenuated Aβ-induced apoptosis and lipid peroxidation in PC12 cells. The effects of L-ergothioneine on the cytotoxicity induced by the nitric oxide donor sodium nitroprusside (SNP) and the peroxynitrite-generating 3-morpholinosydnonimine chlorhydrate (SIN-1) were compared. L-ergothioneine exhibited a concentration-dependent protection of SIN-1-dependent cell death but not that mediated by SNP, suggesting that it is a potent scavenger of peroxynitrite. The transfection of PC12 cells with bcl-2 amplified the L-ergothioneine dependent-rescue of these cells from apoptotic death induced by Aβ. These results, shown in Example 2 below, suggest that L-ergothioneine could modulate oxidative and/or nitrosative neuronal cell death caused by Aβ and may have preventive or therapeutic potential against AD.

6-Hydroxydopamine (OHDA) Lesion Model and Effects of L-Ergothioneine

Much attention has been focused on the in vitro characterization of plant-derived antioxidants. For in vivo considerations, the question of bioavailability and the fate of metabolites of the antioxidant components must be considered. Thus, for the development of therapeutic strategies to prevent progressive neuronal loss based on antioxidant activity, the antioxidant must be able to cross the blood brain barrier and occur at the respective brain region for neuroprotection.

Example 3 below reports the first study to provide evidence that L-ergothioneine reduced the loss of TH+ cells after 6-OHDA lesion in the 6-OHDA lesion rat model. The 6-OHDA lesion rat model fufills the construct validity of Parkinson's disease in that it shares similar biochemical features and the loss of TH+ cells is progressive and dose-dependent (Perese et al. (1989) Brain Res. 494:285-293). The precise mechanism of the neuronal loss due to 6-OHDA is not yet clarified, but there are suggestions that 6-OHDA-dependent oxidative stress inside the neurons may be causing cell death (Ferber et al. (2001a) Neuroreport 12:1155-1159 and Ferber et al. (2001b) J. Neurochem. 78:509-514, both of which publications are herein specifically incorporated by reference in their entirety). The 6-OHDA-induced neuronal death might involve the activation of c-Jun N-terminal kinases (JNK) and extracellular signal-regulated protein kinases (ERK) (Dluzen (2000) J. Neurocytol. 29:387-399, Choi et al. (1999) J. Neuroscience 57:86-94, and Kulich et al. (2001) J. Neurochem. 77:1058-1066, each of which publication is herein specifically incorporated by reference in its entirety). The decrease in the number of TH+ cells after 8 μg of 6-OHDA seen in this study was consistent and comparable to our earlier study (Datla et al. (2001), Neuroreport 12:3871). L-ergothioneine pre-treatment protected the TH+ cell loss.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Example 1

### Assessment of the Effect of L-Ergothioneine in the NMDA Retinal Mo

Materials and Methods

L-Ergothioneine was obtained from Oxis Health Products, Portland Oreg., USA. NMDA and other biochemical were of the highest purity available and purchased from Sigma-Aldrich Chemical Company, UK.

Young adult female Sprague-Dawley rats were used in the present experiments. The animals were supplied by Harlan, England and maintained in the Comparative Biology Unit at Charing Cross Hospital Campus, Imperial College. Animal procedures used were in accordance to regulations of Home Office, UK. The animals were divided into four groups. The first group consists of 6 normal rats that received no treatment. A further 9 animals were anesthetized with. Hypnorm™ (0.02 mg of fentanyl citrate and 0.54 mg fluanisone/100 g body weight) and Hypnovel™ (0.27 mg midazolam/100 g body weight) before they received unilateral intravitreal injection of 5 μl of 4 mM NMDA to the vitreous body of the left eyes, with the uninjected right eyes served as controls. Six of the experimental animals injected with NMDA received an additional intraperitoneal injection of L-ergothioneine 0.2 ml of 70 mg/ml (n=3), or phosphate buffer saline (PBS) as vehicle control 24 h and 30 min before injection of NMDA.

Further intraperitoneal injection of L-ergothioneine, or PBS was performed at 1 h, 24 h, 48 h and 72 h time points, and 3 injections per week for another three weeks. Six weeks after injection of NMDA, all animals were anaesthetized deeply again and perfused with physiological saline followed by 4% paraformaldehyde in phosphate buffer (pH 7.4). The eyeballs were collected in the same fixative and post-fixed for another half an hour before the retinas were dissected out in PBS. For each retina, four radial cuts were made before the retinas were flatly mounted onto gelatin-coated slides, and air-dried slowly in a moist chamber for 2-3 days.

The retinal whole-mounts were then stained for cresyl violet and cover slipped. Analysis was performed under a Wild microscope equipped with a camera lucida drawing tube. The number of retinal neurons in the retinal ganglion cell layer was counted and cell sizes measured at a magnification of 300× and in an area of 150×150 μm in the central, intermediate and peripheral parts of the four retinal quadrants. The neurons counted were divided into two groups with somata smaller than 6 μm, or equal to or larger than 6 μm in diameter. The majority of larger neurons are retinal ganglion cells while smaller somata are primarily non-ganglion cells or displaced amacrine cells (Perry (1981) Neuroscience 6:931-944). The numbers of cells were: counted in a total of 12 fields of individual retinas an analyzed statistically. The data is expressed as mean±S.E.M. Differences between values were compared by one-way analysis of variance (ANOVA).

In a separate series of experiments, eyeballs obtained from 3 normal rats and 3 rats 24 hours after intravitreal injection of NMDA were dissected out after perfusion with 4% paraformaldehyde, cryoprotected in 30% sucrose and cut on a cryostat at a thickness of 20 μm. Alternate sections were collected on gelatin-coated slides and stained for cresyl violet to reveal the cytoarchitecture of the retina or reacted immunocytochemically for amyloid precursor protein (APP) (Sigma-Aldrich, UK 1:800) and visualized using the Avidin-biotin complex method (Vector Laboratories, UK).

Results

There was a 58% reduction in total cell numbers in the retina of animals that received intravitreal injection of NMDA followed by treatment with phosphate buffered saline (PBS control group). This reduction was particularly apparent in the larger cells with an 81% loss of ganglion cells and a 43% reduction in non-ganglion cells. In contrast, there was a loss of only 15% of ganglion cells and 8% of non-ganglion cells in the uninjected retinas (FIGS. 4-5). In L-ergothioneine treated animals, there was a loss of 44% of ganglion cells and 31% of the small or non-ganglion cells. The uninjected control eyes from these animals showed a loss of 7% and 4% of these populations (FIG. 5). These results demonstrate a significant neuroprotective effect of L-ergothioneine.

### Example 2

### Assessment of the Effect of L-Ergothioneine on Cytotoxicity and Apoptotic Cell Death Induced by β-Amyloid in PC12 Cells

### A. Effect of L-Ergothioneine on β-Amyloid Cytotoxicity of PC12 Cells

Materials

MTT [3-(4,5-dimethylthiazol-2-yl)-2,5-diphenyltetrazolium bromide] and sodium nitroprusside (SNP) were purchased from Sigma Chemical Co. (St. Louis, Mo., USA). beta-Amyloid peptide (Aβ₂₅₋₃₅) was obtained from Bachem Inc. (Torrance, Calif., USA). Aβ₂₅₋₃₅ was dissolved in deionized distilled water at a concentration of 1 mM and stored at −20° C. until used. The stock solutions were diluted to desired concentrations immediately before use and added to culture medium without the aging procedure. We note that both fresh and aged preparations of Aβ₂₅₋₃₅ have similar cytotoxic effects in PC12 cells. Dulbecco's modified Eagle's medium (DMEM), fetal bovine serum, horse serum, nutrient mixture Ham's F-12 and N-2 supplement were provided from Gibco BRL (Grand Island, N.Y., USA). 3-Morpholinosydnonimine chlorhydrate (SIN-1) was a product of Biomol Research Lab, Inc. (Plymouth Meeting, Pa., USA). Tetramethylrhodamine ethyl ester (TMRE) and dihydrorhodamine (DHR) 123 were supplied from Molecular Probes, Inc. (Eugene, Oreg., USA) and Fluka Chemie GmnH (Buchs, Switzerland), respectively. Synthetic EGT was obtained from OXIS International (Portland, Oreg., USA).

Cell Culture

PC12 cells were maintained in DMEM supplemented with 10% heat-inactivated horse serum and 5% fetal bovine serum at 37° C. in a humidified atmosphere of 10% CO₂/90% air. All cells were cultured in poly-D-lysine coated culture dishes. The medium was changed every other day, and cells were plated at an appropriate density according to each experimental scale. After 24 h subculture, cells were switched to serum-free N-2 defined medium for treatment. For determination of cell viability, PC12 cells were initially plated at a density of 4×10⁴ cells/300 μl in 48-well plates, and the cell viability was determined by the conventional MTT reduction and the lactate dehydrogenase (LDH) release assay as described below.

MTT Dye Reduction Assay

The MTT assay is a sensitive measurement of the normal metabolic status of cells, particularly that of mitochondria, which reflects early cellular redox changes. After incubation, cells were treated with the MTT solution (final concentration, 1 mg/ml) for 2 h. The dark blue formazan crystals formed in intact cells were dissolved in DMSO, and absorbance at 570 nm was measured with a microplate reader. Results were expressed as the percentage (%) of MTT reduction, assuming that the absorbance of control cells was 100%.

LDH Release Assay

This assay measures the leakage of the soluble cytoplasmic LDH enzyme into the extracellular medium due to cell lysis. PC12 cells were plated at the same density as for the MTT assay described above. The amount of lactate was measured by monitoring the oxidation of L-lactic acid by NAD⁺ in the presence of LDH to pyruvate. The culture media were transferred to 96-well plate and incubated with 1 mg/ml β-NAD⁺ in pyruvate substrate solution at 37° C. for 30 min. After additional incubation at room temperature for 20 min with a color reagent (2,4-dinitrophenylhydrazine), the reaction was stopped by addition of 0.4 N NaOH. The changes in absorbance were determined at 450 nm using a spectrophotometric microplate reader.

Results

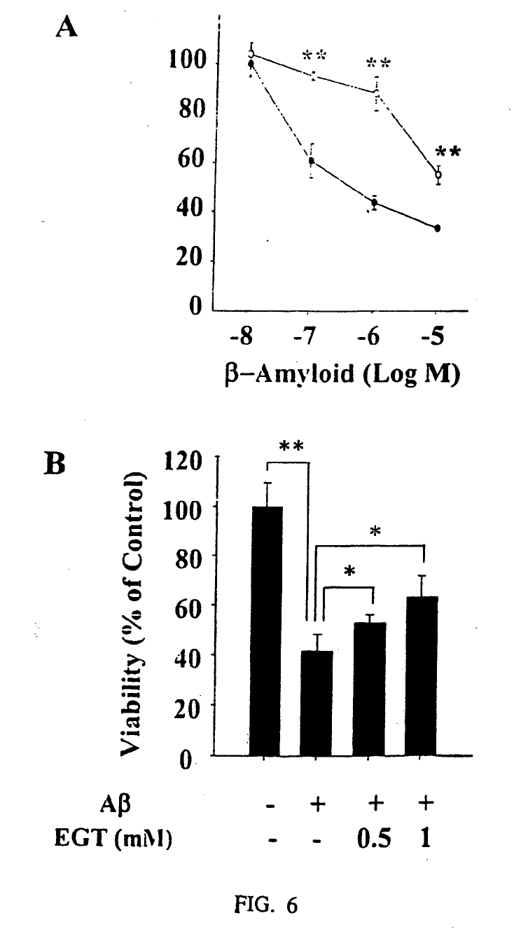

The cytotoxicity of Aβ was initially assessed by the conventional MTT by determining the percentage (%) of MTT reduction after incubation of PC12 cells for 36 h with increasing concentrations of Aβ₂₅₋₃₅. Aβ₂₅₋₃₅ decreased the cell viability concentration dependently, and its cytotoxic effect was inhibited by 1 mM EGT (FIG. 6A). In order to correlate the MTT reductive activity with cell death and subsequent protection by EGT, cellular damage was evaluated quantitatively by the amount of LDH released into media in the presence and absence of EGT. The cytoprotective effect of EGT was verified by its ability to reduce the LDH release in the Aβ₂₅₋₃₅-treated PC12 cells (FIG. 6B).

### B. Effect of L-Ergothioneine on β-Amyloid Induced Apoptosis in PC12 Cells

Terminal Deoxynucleotidyl Transferase-Mediated dUTP Nick End-Labeling (TUNEL) Procedure

The commercially available in situ death detection kit (Boehringer Mannheim product, Manheim, Germany) was utilized to detect DNA fragmentation. The PC12 cells (5×10⁵ cells/1.5 ml in chamber slide) were fixed for 30 min in 10% neutral buffered-formalin solution at room temperature. Endogenous peroxidase was inactivated by incubation with 0.3% (v/v) hydrogen peroxide in methanol for 30 min at room temperature and further incubated in a permeabilizing solution (0.1% sodium citrate and 0.1% Triton X-100) for 2 min at 4° C. The cells were labeled by incubation with the TUNEL reaction mixture for 60 min at 37° C. followed by labeling with peroxidase-conjugated anti-fluorescein anti-goat antibody (Fab fragment) for additional 30 min. After staining with diaminobenzidine for 10 min, cells were rinsed with phosphate-buffered saline (PBS) and mounted with 50% glycerol.

Measurement of the Mitochondrial Membrane potential (ΔΨm)

To measure the mitochondrial membrane potential (ΔΨm), the lipophilic cationic probe TMRE was used. After treatment with Aβ₂₅₋₃₅ (25 μM) for 24 h in the presence or absence of EGT, cells (1×10⁴ cells/1 ml in 4-well chamber) were rinsed with PBS, and TME (150 nM) was loaded. After 30 min incubation at 37° C., cells were examined under a confocal microscope (LEICA TCS SP). TMRE exhibits potential-dependent accumulation in mitochondria, which was detectable by the fluorescence excitation at 488 nm and emission at 590 nm.

Western Blot Analysis

After treatment, cells (1×10⁷ cells/7 ml in 100φ dish) were collected and washed with PBS. After centrifugation, cell lysis was carried out at 4° C. by vigorous shaking for 15 min in RIPA buffer (150 mM NaCl, 1% NP-40, 0.5% sodium deoxycholate, 0.1% SDS, 50 mM Tris-HCl (pH 7.4), 50 mM glycerphosphate, 20 mM NaF, 20 mM EGTA, 1 mM DTT, 1 mM Na₃VO₄ and protease inhibitors). After centrifugation at 15,000 rpm for 15 min, supernatant was separated and stored at −70° C. until use. The protein concentration was determined by using the bicinchrinic acid (BCA) protein assay kit (Pierce, Rockford, Ill., USA). After addition of sample loading buffer, protein samples were electrophoresed on a 12.5% SDS-polyacrylamide gel. Proteins were transferred to polyvinylidene difluoride blots at 300 mA for 3 h. The blots were blocked for 1 h at room temperature in fresh blocking buffer (0.1% Tween-20 in Tris-buffered saline, pH 7.4 containing 5% non-fat dried milk). Dilutions (1:1000) of primary anti-poly(ADP-ribose)polymerase (PARP), anti-Bcl-X_{L} and anti-Bax antibodies were made in PBS with 3% non-fat dry milk. Following three washes with PBST (PBS and 0.1% Tween-20), the blots were incubated with horseradish peroxidase-conjugated secondary antibodies in PBS with 3% non-fat dry milk for 1 h at room temperature. The blots were washed again three times in PBST buffer, and transferred proteins were incubated with ECL substrate solution (Amersham Pharmacia Biotech, Inc., Piscataway, N.J., USA) for 1 min according to the manufacturer's instructions and visualized with x-ray film.

Results

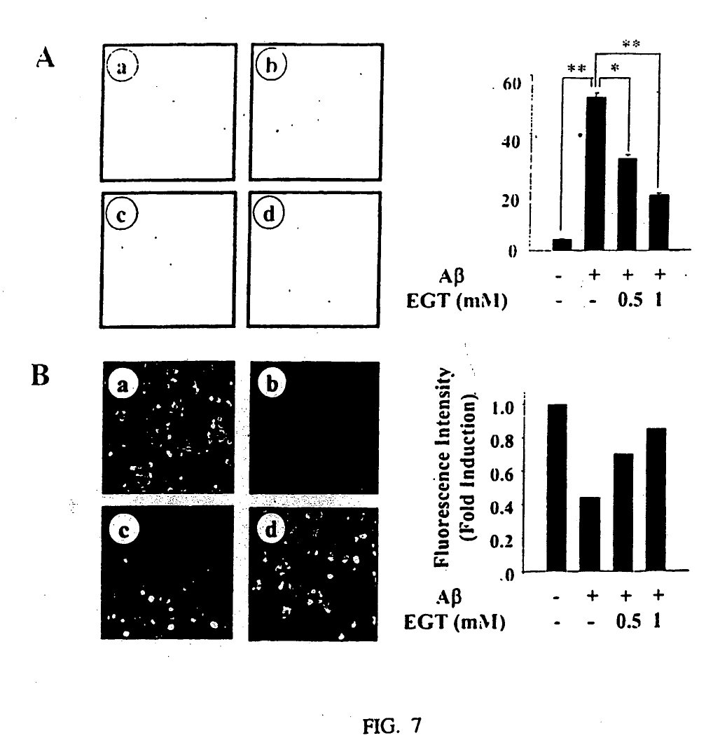

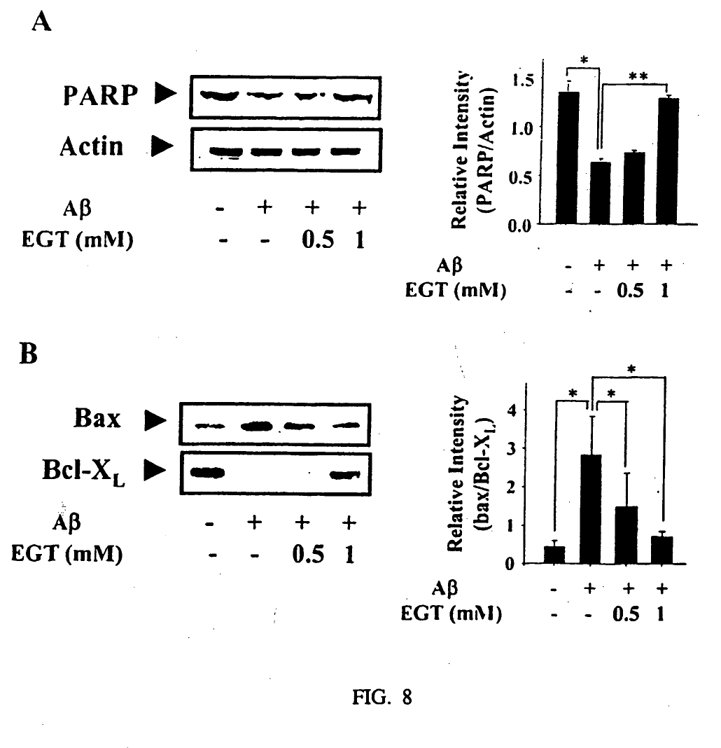

PC12 cells treated with 25 μM Aβ underwent apoptosis as determined by positive terminal end labeling (TUNEL) that detects DNA fragmentation in situ. In this histochemical analysis, the appearance of intensely stained nucleus is indicative of terminal incorporation of labeled dUTP into the 3′-end of fragmented DNA derived from apoptotic nuclei. EGT, at 0.5 mM or 1 mM, lowered the proportion of TUNEL-positive cells (FIG. 7A). Besides the nuclear DNA fragmentation, more recently, mitochondria is recognized as a key step in apoptosis. Mitochondria undergoes major changes in membrane integrity before classical signs of cell death become manifest. These changes include both the inner and the outer mitochondrial membranes, leading to the dissipation of the transmembrane potential and/or permeability changes which release of soluble intermembrane proteins through the outer membrane. When PC12 cells were exposed to Aβ₂₅₋₃₅ (25 μM), the mitochondrial transmembrane potential (ΔΨm) was rapidly reduced, as shown by the decrease in red-fluorescence using voltage-sensitive dye TMRE (FIG. 7B). Aβ₂₅₋₃₅-induced dissipation of ΔΨm was significantly blocked by the pretreatment of EGT (FIG. 7B). Aβ₂₅₋₃₅-induced apoptotic cell death was verified by examining the cleavage of PARP. PARP is a 116 kDa nuclear protein which is specifically cleaved by active caspase-3 into 85 kDa apoptotic fragment. Treatment with 25 μM Aβ₂₅₋₃₅ caused cleavage of PARP, which was inhibited by EGT (FIG. 8A). The expression of Bcl-2 family proteins was also examined. The ratio of pro-apoptotic Bax and the anti-apoptotic Bcl-2 is considered as a molecular rheostat determining cell survival/death. Since Bcl-2 was barely detectable in PC12 cells, we alternatively measured the levels of Bcl-X_{L} that is structurally and functionally-analogous to Bcl-2. As illustrated in FIG. 8B, Aβ treatment led to increased expression of proapoptotic Bax with concomitant decrease in the level of anti-apoptotic protein BCl-X_{L}. EGT treatment substantially reduced the ratio of Bax to Bcl-X_{L}.

### C. Effect of L-Ergothioneine on β-Amyloid Induced Nitrosative Damage in PC12 Cells

Measurement of Intracellular Peroxynitrite Formation

To monitor intracellular formation of peroxynitrite, the fluorescent probe DHR123 was used. DHR123 is lipophilic and readily diffuses across cell membranes. Upon oxidation of DHR to fluorescent rhodamine, one of the two covalent amino groups tautomerizes to a changed imino, effectively trapping rhodamine within cells. DHR is not oxidized by nitric oxide (NO) but peroxynitrite effectively oxidizes it. After treatment with Aβ₂₅₋₃₅ (25 μM) for 36 h in the presence or absence of L-ergothioneine, cells (1×10⁴ cells/1 ml in 4-well chamber slide) were rinsed with saline A, and 10 μM DHR in saline A containing 5% fetal bovine serum was loaded. After 20 min incubation at 37 C, cells were examined under a confocal microscope equipped with an argon laser (488 nm; 200 mW). To quntitate peroxynitrite generation in response to Aβ₂₅₋₃₅, total peroxynitrite production (basal+incease) was divided by basal peroxynitrite generation. Changes in fluorescence intensity are expressed as a percentage of the control.

Assessment of Lipid Peroxidation

The extent of lipid peroxidation in PC12 cells treated with Aβ₂₅₋₃₅ was assessed using the commercially available calorimetric assay kit BIOXYTECH LPO-586 (OXIS Research, Portland, Oreg.). After exposure to 50 μM Aβ₂₅₋₃₅ in the presence or absence of L-ergothioneine at 37° C. for 24 h, PC12 cells were harvested and homogenized in 20 mM Tris-HCl buffer (pH 7.4), containing 0.5 mM butylated hydroxytoluene to prevent sample oxidation. After centrifugation, 3.25 volumes of diluted R1 reagent (10.3 mM N-methyl-2-phenylindole in acetonitrile) was added to the supernatant, followed by gentle vortex mixing following the addition of 0.75 ml of 37% (v/v) HCl, the mixtures were incubated at 45° C. for 60 min. After cooling and centrifugation, the absorbance of the clear supernatant was read at 590 nm. The protein concentration was determined using the BCA protein assay kit.

Results

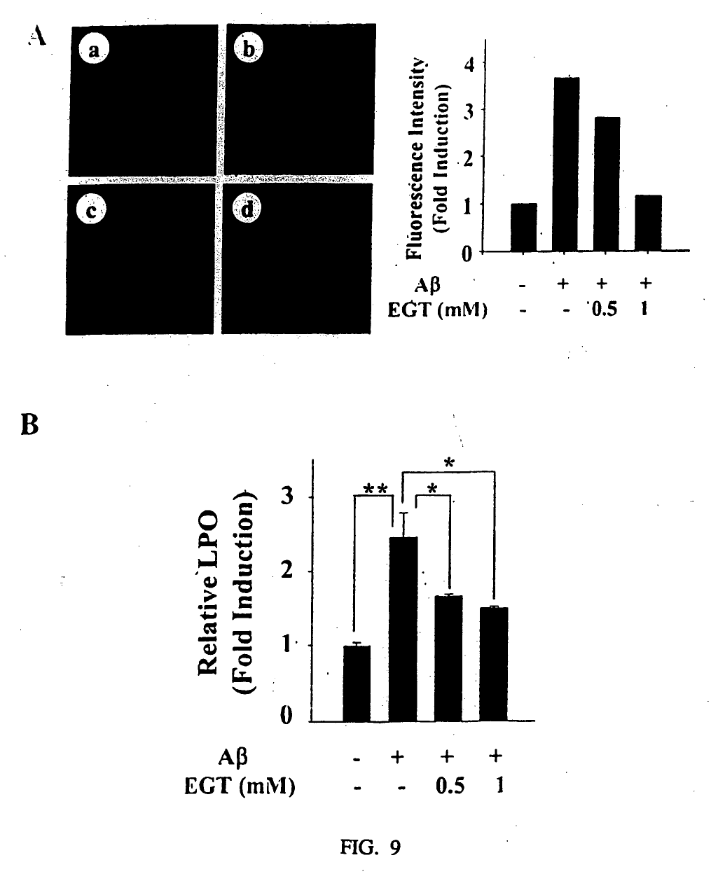

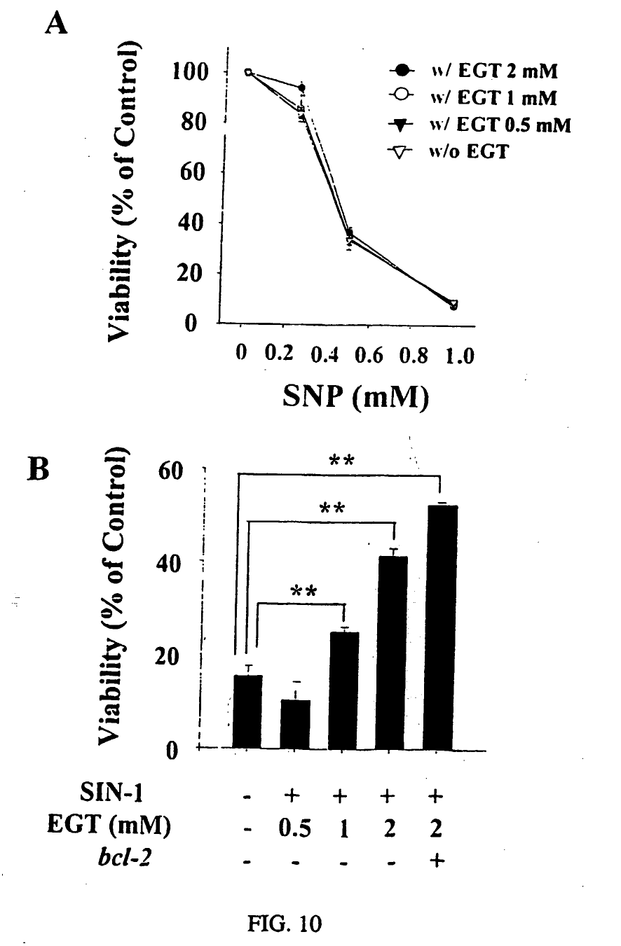

The effect of L-ergothioneine on the Aβ-induced intracellular peroxynitrite generation was measured using DHR dye, which is rapidly oxidized by the peroxynitrite to fluorescent rhodamine. PC12 cells treated with 25 μM Aβ₂₅₋₃₅ displayed intense fluorescence after staining with DHR, and intracellular peroxynitrite formation resulting from Aβ₂₅₋₃₅ treatment was significantly reduced when L-ergothioneine was present in the media (FIG. 9A). Aβ₂₅₋₃₅ can cause nitrosative damage through generation of reactive nitrogen species (RNS) and modulation of redox sensitive signals that results in the disruption of phospholipid bilayer of neuronal cells. PC12 cells treated with Aβ₂₅₋₃₅ underwent peroxidation of its lipid bilayer leading to increased levels of lipid peroxides (FIG. 9B). Pretreatment with L-ergothioneine for 30 min resulted in concentration dependent inhibition of lipid peroxidation. (FIG. 9B). Moreover, L-ergothioneine selectively protected against cytotoxicity induced by the peroxynitrite releasing compound SIN-1 (FIG. 10B), while it failed to attenuate the cell death mediated by the NO donor SNP (FIG. 10A), indicating that L-ergothioneine an effective scavenger of peroxynitrite.

### D. Assessment of the Effects of L-Ergothioneine on β-Amyloid Induced Activation of NF-κB

Preparation of Nuclear Extracts

To explore the molecular mechanisms underlying the protective effect of L-ergothioneine against Aβ₂₅₋₃₅-induced nitrosative cell death, the activation of NF-κB was assessed by EMSA using an oligonucleotide containing a consensus κB binding element. After treatment with 25 μM Aβ₂₅₋₃₅ for 1 h in the absence or presence of L-ergothioneine, PC12 cells (1×10⁷ cells/7 ml in 100φ dish) were washed with PBS, centrifuged, and resuspended in ice-cold isotonic buffer A [10 mM HEPES, pH 7.9, 1.5 mM MgCl₂, 10 mM KCl, 0.5 mM dithiothreitol (DTT) and 0.2 mM phenylmethylsulfonyl fluoride (PMSF)]. Following incubation in an ice bath for 10 min, cells were centrifuged again and resuspended in ice-cold buffer C containing 20 mM HEPES (pH 7.9), 20% glycerol, 420 mM NaCl₂, 1.5 mM MgCl₂, 0.2 mM EDTA, 0.5 mM DTT and 0.2 mM PMSF followed by incubation at 0° C. for 20 min. After vortex-mixing, the resulting suspension was centrifuged, and the supernatant was stored at −70° C. for the NF-κB DNA binding assay. The protein concentration was determined by using the BCA protein assay kit.

Electrophoretic Mobility Shift Assay (EMSA) for Determining the NF-κB DNA Binding Activity

Synthetic double strand oligonucleotide containing the NF-κB binding domain was labeled with [γ-³²P]ATP using T4 polynucleotide kinase and separated from unincorporated [γ-³²P]ATP by gel filtration using a nick spin column (Phamacia Biotech, Bjorkgatan, Sweden). Prior to addition of the radio-labeled oligonucleotide (100,000 cpm), 10 μg of the nuclear extract was kept on ice for 15 min in gel shift binding buffer [4% glycerol, 1 mM EDTA, 1 mM DTT, 100 mM NaCl, 10 mM Tris-HCl, (pH 7.5) and 0.1 mg/ml sonicated salmon sperm DNA]. DNA-protein complexes were resolved by 6% non-denaturating polyacrylamide gel at 200 V for 2 h followed by autoradiography.

Immunocytocheistry of p65

For immunocytochemistry, PC12 cells (10⁵ cells/800 μl in chamber slide) were fixed for 30 min in 10% neutral buffered-formalin solution at room temperature. The cells were blocked for 1 h at room temperature in fresh blocking buffer (5.5% normal goat serum in TBST). Dilutions (1:100) of primary anti-nitrotyrosine antibody were made in TBS with 3% BSA. Following three washes with TBST, the cells were incubated with FITC-conjugated secondary antibodies in TBS with 3% BSA for 1 h at room temperature. Cells were washed again three times in TBST buffer and incubated with propidium iodide for 10 min for the staining of nucleus. Cells were rinsed with TBS and examined under a confocal microscope.

Statistical Analysis

Data were expressed as means±SD, and statistical analysis for single comparison was performed by Student's t-test. The criterion for statistical significance was P<0.05.

Results

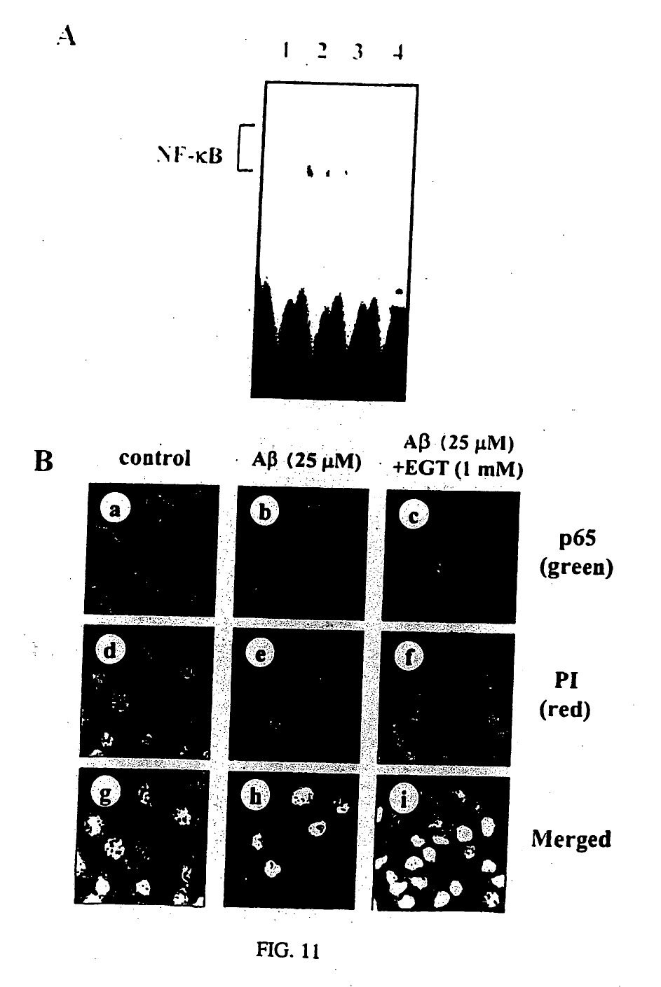

Treatment of PC12 cells with Aβ₂₅₋₃₅ caused a transient increase in NF-κB DNA binding, which was inhibited by EGT pretreatment (FIG. 11A). To further verify the inhibitory effect of EGT on Aβ₂₅₋₃₅-induced activation of NF-κB, we measured the nuclear translocation of p65, a functionally active subunit of NF-κB in PC12 cells, by immunocytochemistry using anti-p65 antibo and propidium iodide (FIG. 11B).

In assessing the neuroprotective effects of L-ergothioneine on β-Amyloid induced damage in PC12 cells, the following results were observed. Aβ-induced apoptotic death via nitrosative stress PC12 cells was suppressed by treatment with L-ergothioneine. Cytotoxicity induced by Aβ by the conventional MTT reduction assay was used in this assessment. Aβ caused a decrease in MTT reduction in PC 12 cells, which was partly restored in the presence of EGT. The protective effect of L-ergothioneine on Aβ₂₅₋₃₅-induced cytotoxicity was confirmed using the LDH release assay.

In addition, Aβ-induced intracellular formation of peroxynitrite was attenuated by L-ergothioneine, as revealed by reduced distribution of the DHR fluorescent dye in cells pretreated with this compound. Furthermore, EGT exhibited a concentration-dependent protection of SIN-1-dependent cell death but not the SNP-mediated cytotoxicity, suggesting that L-ergothioneine is a potent scavenger of peroxynitrite. SIN-1 only generates peroxynitrite through a sequential release c [symbol] superoxide and nitric oxide and their diffusion-limited reaction. Thus, one means by which L-ergothioneine exhibits its neuroprotective effects may be by way of its inhibitory effects on peroxynitrite production.

Furthermore, Aβ₂₅₋₃₅ treatment caused the impairment of mitochondrial membrane potential, the decreased antiapoptotic Bcl-X_{L}/proapoptotic Bax ratio, and the cleavage of PARP. Pretreatment of cells with L-ergothioneine attenuated these biochemical changes associated with Aβ-induced apoptosis.

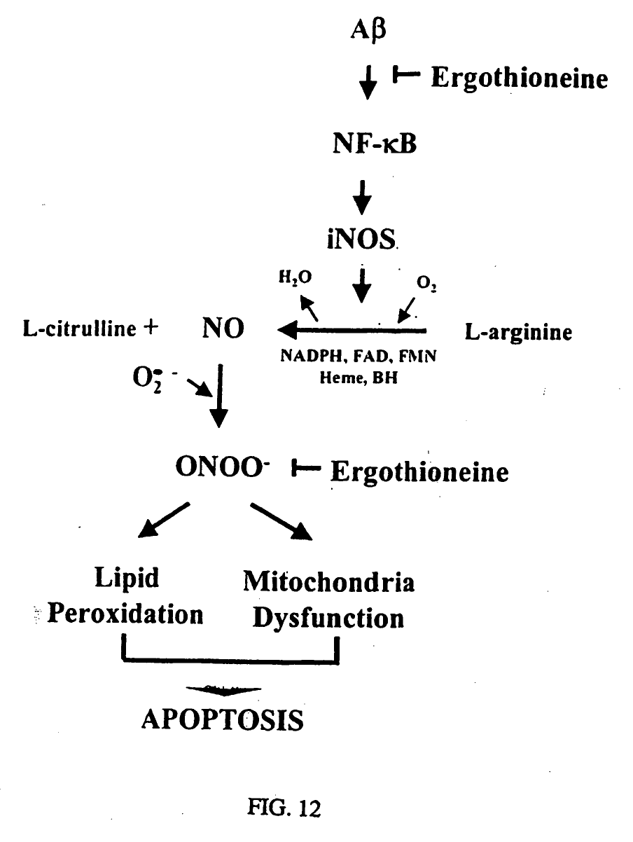

Aβ₂₅₋₃₅ treatment also causes NF-κB activation in PC12 cells, which can be attenuated by L-ergothioneine pretreatment. A proposed mechanism for the neuroprotective effects of L-ergothioneine is shown in FIG. 12.

### Example 3

### Assessment of the Neuroprotective Effects of L-ergothioneine in the 6-OHDA Model

Male Sprague-Dawley rats with starting weights 225±25 g, were housed in groups of 3 with free access to food and water, under controlled temperature (21° C.±1° C.) and a 12 hour light/dark cycle (light on 07.00 hrs). All scientific procedures were carried out with the approval of the Home Office, U.K. Rats were administered, by gavage, 70 mg/kg of ergothioneine or vehicle (sterile distilled water) daily for 4 days (n=6 per group). On the 4^{th} day, 1 hr after L-ergothioneine or vehicle administration, rats were anaesthetized with small animal Immobilon® (0.04 ml/rat, i.m.), and 6-OHDA (5 μg dissolved in 4 μl of 0.1% ascorbic acid/saline solution) was injected onto median forebrain bundle (stereotactic co-ordinates: 2.2 mm anterior, +1.5 lateral from bregma and −7.9 ventral to dura with ear bars 5 mm below incisor bars (Datla et al. (2001) Neuroreport 12:3871, which reference is herein specifically incorporated by reference in its entirety). One week after 6-OHDA lesioning, rats were killed by cervical dislocation and the brains were dissected out immediately. A coronal section was made at the level of hypothalamus and fore brain, and hind brain parts were separated. Hind-brain was fixed for 7 days in 4% paraformaldehyde, then cryo-protected with 30% sucrose solution for 2-3 days and used for tyrosine hydroxylase (TH) immuno-staining as described by Datla et al. (2001) supra. Briefly, TH was immuno-stained by incubating the 20 μm fixed coronal free-floating sections with polyclonal rabbit anti-TH (1:3000, Chemicon, U.K.) followed by biotinylated anti-rabbit IgG and avidin/biotin complex (Vector Lab, U.K.). The TH immuno-complex was then visualized by diaminobenzidine (DAB) and H₂O₂. Images of TH positive cells (TH+ cells) were captured by a Xillix CCD digital camera and counted automatically (Image Proplus, Datacell, U.K.). The number of TH+ cells in the substantia nigra on the control side was compared with the lesioned side by averaging the cells in 5 different levels (Datla et al. (2001) supra). From the fore brain, lesioned and control striata were dissected out and assayed for DA and its metabolites, DOPAC and HVA, by HPLC-electrochemical detection (Datla et al. (2001) supra).

Results

After 28 days of oral administration of L-ergothioneine, and injection of 6-hydroxydopamine (6-OHDA), the integrity and functionality of nigro-striatal dopaninergic pathways in the 6-OHDA lesion PD model were assessed. The number of dopaminergic cells in the substantia nigra was determined by immuno-staining for tyrosine hydroxylase and measuring dopamine levels in the striatum by HPLC.

The number of TH+ cells on the control side of the brain of both vehicle and L-ergothioneine treated groups was comparable. Overall effects of lesion and L-ergothioneine treatment on TH+ cells were analyzed by ANOVA with lesion as within subject factor and L-ergothioneine treatment as between subject factor. There were significant effects of lesioning (Data<0.001) and L-ergothioneine by lesion (Data p<0.01). Individual group comparisons by Student's t-test showed that lesioning significantly reduced TH+ cells (p<0.005; paired Student's t-test) in both vehicle and L-ergothioneine treated groups. However, the reduction in the number of TH+ cells in vehicle treated group was significantly higher (63% reduction) than in the L-ergothioneine treated group (46% reduction) (p<0.0005; unpaired Student's t-test). Thus, L-ergothioneine demonstrated significant improvement (approximately 20%) in terms of neuroprotection over the controls.

What is claimed is:

1. A method of protecting a mammalian central nervous system (CNS) cell from damage, comprising administering a therapeutically effective amount of L-ergothioneine to a mammal in need thereof.

2. The method of claim 1, wherein the CNS cell is a neuronal cell.

3. The method of claim 2, wherein the neuronal cell is a ganglion and a non-ganglion cell.

4. The method of claim 2, wherein the neuronal cell is one or more of a cholinergic, a dopaminergic and a GABAergic neurons.

5. The method of claim 2, wherein the neuronal cell is a dopaminergic neuron.

6. The method of claim 5, wherein the dopaminergic neurons are tyrosine hydroxylase positive (TH+) cells of the substantia nigra.

7. The method of claim 1, wherein the damage results from exposure to an oxidant.

8. The method of claim 7, wherein the oxidant is selected from the group consisting of singlet oxygen, hydrogen peroxide, nitric oxide, hypochlorous acid, hydroxyl radicals, peroxyl radicals, and metalloenzymes.

9. The method of claim 1, wherein the damage results from exposure to a cytokine.

10. The method of claim 9, wherein the cytokine is tumor necrosis factor-α (TNF-α) or gamma interferon.

11. The method of claim 1, wherein the damage results from exposure to a neurotoxic compound.

12. The method of claim 11, wherein the neurotoxic compound is selected from the group consisting of glutamate, a glutamate analog, and an anticancer compound.

13. The method of claim 1, wherein the damage results from the presence of a neurodegenerative disease.

14. The method of claim 13, wherein the neurodegenerative disease is selected from the group consisting of Alzheimer's disease, multiple sclerosis, Down's syndrome, amyotropic lateral sclerosis, Parkinson's disease, traumatic brain injury, acute and chronic spinal cord injury, macular degeneration, HIV/AIDS, optic neuropathies and-retinopathies.

15. The method of claim 1, wherein the mammal is a human being.

16. The method of claim 1, wherein L-ergothioneine is administered as a dietary supplement.

17. The method of claim 16, wherein the dietary supplement is in the form of an oral capsule, tablet, or suspension.

18. The method of claim 1, wherein L-ergothioneine is administered in combination with a second anti-oxidant.

19. The method of claim 18, wherein the second anti-oxidant is vitamin C or vitamin E.

20. The method of claim 1, wherein L-ergothioneine is administered in combination with agents that aid in protection of neuronal cells, or agents that aid in cellular proliferation and/or tissue regeneration and/or remyelination.

21. The method of claim 20, wherein said agents that aid in protection of neuronal cells, or agents that aid in cellular proliferation and/or tissue regeneration and/or remyelination are selected from the group consisting of small synthetic organic compounds, proteins, peptides, polypeptides, nucleic acids, polynucleotides, antisense oligonucleotides, and antibodies.

22. The method of claim 20, wherein said agent is a ROS scavenger selected from the group consisting of coenzyme Q, vitamin E, vitamin C, pyruvate, melatonin, niacinamide, N-acetylcysteine, GSH, and nitrones.

23. The method of claim 20, wherein said agent is selected from the group consisting of neurotrophic factors, ligands that bind to and activate receptor protein kinases, agonist ligands for integrin receptors, receptor mimics, members of the immunoglobulin superfamily and remyelinating antibodies.

24. A method of treating or ameliorating damage to a mammalian central nervous system (CNS) cell from a neurodegenerative disease, comprising administering a therapeutically effective amount of L-ergothioneine to a mammal in need thereof.

25. The method of claim 24, wherein administration of L-ergothioneine is chronic.

26. The method of claim 24, wherein the neurodegenerative disease is selected from the group consisting of Alzheimer's disease, multiple sclerosis, Down's syndrome, amyotropic lateral sclerosis, Parkinson's disease, traumatic brain injury, acute or chronic spinal cord injury, macular degeneration, HIV/AIDS, optic neuropathies and retinopathies.

27. The method of claim 24, wherein the CNS cell is a neuronal cell.

28. The method of claim 27, wherein the neuronal cell is a ganglion and a non-ganglion cell.

29. The method of claim 27, wherein the neuronal cell is one or more of a cholinergic, a dopaminergic and a GABAergic neurons.

30. The method of claim 27, wherein the neuronal cell is a dopaminergic neuron.

31. The method of claim 30, wherein the dopaminergic neurons are tyrosine hydroxylase positive (TH+) cells of the substantia nigra.

32. A screening method for identifying compounds capable of protecting central nervous system (CNS) cells from damage, comprising (a) treating retinal neurons to a neurotoxic agent with and without treatment with a test compound; and (b) determining the effect of the test compound on a retinal neuron population, wherein a test compound capable of increasing cell survival is identified as a neuroprotective agent.

33. A screening method for identifying compounds capable of protecting central nervous system (CNS) cells from damage, comprising (a) treating dopaminergic neurons with 6-OHDA with and without treatment with a test compound; and (b) determining the effect of the test compound on the dopaminergic neuron population, wherein a test compound capable of increasing cell survival is identified as a neuroprotective agent.

34. The method of claim 1, wherein the administering is by oral administration or by intravitreal, intramuscular, intraperitoneal, intrathecal, intraventricular or intracranial injection.

35. A pharmaceutical composition comprising a therapeutically effective amount of L-ergothioneine and a pharmaceutically acceptable carrier.

36. The pharmaceutical composition of claim 35, further comprising a therapeutically effective amount of an agent that aids in protection of neuronal cells, or an agent that aids in cellular proliferation and/or tissue regeneration and/or remyelination.

37. The pharmaceutical composition of claim 36, wherein the agent is selected from the group consisting of small synthetic organic compounds, proteins, peptides, polypeptides, nucleic acids, polynucleotides, antisense oligonucleotides, and antibodies.

38. The pharmaceutical composition of claim 37, wherein the agent is a Reactive Oxygen Species (ROS) or a Reactive Nitrogen Species (RNS) scavenger.

39. The pharmaceutical composition of claim 38, wherein the ROS scavenger is selected from the group consisting of coenzyme Q, vitamin E, vitamin C, pyruvate, melatonin, niacinamide, N-acetylcysteine, GSH, and nitrones.

40. The pharmaceutical composition of claim 37, wherein the agent is selected from the group consisting of neurotrophic factors, ligands that bind to and activate receptor protein kinases, agonist ligands for integrin receptors, receptor mimics, members of the immunoglobulin superfamily and remyelinating antibodies.
